(19) United States
(12) Patent Application Publication
(10) Pub. No.: US 2024/0033201 A1
(43) Pub. Date: Feb. 1, 2024

(54) COMPOSITION COMPRISING POLYION COMPLEX PARTICLE AND FILLER

(71) Applicant: L'OREAL, PARIS (FR)

(72) Inventors: Shinobu MITSUDA, Kawasaki-shi (JP); Hiroyuki OGATA, Kawasaki-shi (JP)

(21) Appl. No.: 18/256,580

(22) PCT Filed: Dec. 10, 2021
(86) PCT No.: PCT/JP2021/046652

§ 371 Date: Jun. 8, 2023

(30) Foreign Application Priority Data
Dec. 14, 2020 (JP) 2020-206664
Jan. 13, 2021 (FR) 2100277

(51) Int. Cl. A61K 8/58; A61Q 19/00; A61K 8/04; A61K 8/73; A61K 8/81

(52) U.S. Cl. CPC A61K 8/585; A61Q 19/008; A61K 8/042; A61K 8/735; A61K 8/817; A61K 2800/95

(57) ABSTRACT

The present invention relates to a composition comprising: (a) at least one particle, comprising at least one cationic polymer and at least one anionic polymer, and at least one non-polymeric acid having two or more pKa values or salt(s) thereof; (b) at least one filler; and (c) water. The composition according to the present invention can provide improved an moisturizing texture, preferably a combination of an improved moisturizing texture and mattifying effect.

### TECHNICAL FIELD

The present invention relates to a composition including polyion complex particle(s) and filler(s), as well as a cosmetic process using the composition.

### BACKGROUND ART

It has been known that shiny or greasy skin due to sebum may emphasize the roughness on the skin. In other words, shiny skin may make skin roughness such as pores and wrinkles more noticeable. Therefore, users of cosmetic products would like to achieve a matte appearance of the skin.

There are some oil-absorbable powders. Such powders may be used in cosmetic products to provide the skin with mattifying effects by absorbing sebum with these powders.

However, oil-absorbing powders often cause undesirable dry or squeaky sensations even in cosmetic products including water. In other words, it is often difficult to use oil-absorbing powders in cosmetic products including water for providing a good moisturizing texture.

The above problem is not limited to only oil-absorbing powders. For example, the use of fillers in cosmetic products may cause a squeaky texture, and it may not be possible to obtain a good moisturizing texture even if the cosmetic products include water.

### DISCLOSURE OF INVENTION

Therefore, there is a need for a composition which includes a filler but can provide an improved moisturizing texture.

Thus, an objective of the present invention is to provide a composition which is capable of providing an improved moisturizing texture, preferably both improved moisturizing texture and mattifying effect.

The above objective of the present invention can be achieved by a composition, comprising:
(a) at least one particle, comprising
   at least one cationic polymer and at least one anionic polymer, and
   at least one non-polymeric acid having two or more pKa values or salt(s) thereof;
(b) at least one filler; and
(c) water.

The cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group.

The cationic polymer may be selected from the group consisting of cyclopolymers of alkyldiallylamine and cyclopolymers of dialkyldiallylammonium such as (co)polydiallyldialkyl ammonium chloride, (co)polyamines such as (co)polylysines, cationic (co)polyaminoacids such as collagen, cationic cellulose polymers, and salts thereof.

The amount of the cationic polymer(s) forming the (a) particle in the composition according to the present invention may be from 0.01% to 15% by weight, preferably from 0.05% to 10% by weight, and more preferably from 0.1% to 5% by weight, relative to the total weight of the composition.

The anionic polymer may be selected from hyaluronic acid and derivatives thereof.

The amount of the anionic polymer(s) forming the (a) particle in the composition according to the present invention may be from 0.01% to 15% by weight, preferably from 0.05% to 10% by weight, and more preferably from 0.1% to 5% by weight, relative to the total weight of the composition.

The non-polymeric acid having two or more pKa values or salt(s) thereof may be an organic acid or salt(s) thereof, preferably a hydrophilic or water-soluble organic acid or salt(s) thereof, and more preferably phytic acid or salts thereof.

The amount of the non-polymeric acid having two or more pKa values or salt(s) thereof forming the (a) particle in the composition according to the present invention may be from 0.01% to 15% by weight, preferably from 0.05% to 10% by weight, and more preferably from 0.1% to 5% by weight, relative to the total weight of the composition.

The amount of the (a) particle in the composition according to the present invention may be from 0.01% to 15% by weight, preferably from 0.05% to 10% by weight, and more preferably from 0.1% to 5% by weight, relative to the total weight of the composition.

The (b) filler may be selected from hydrophilic or hydrophobic oil-absorbing powders. The hydrophobic oil-absorbing powders may be selected from powders of hydrophobic silicas, preferably hydrophobic silica aerogels, and more preferably hydrophobic aerogels of silica silylate.

The amount of the (b) filler(s) in the composition according to the present invention may be from 0.01% to 15% by weight, preferably from 0.05% to 10% by weight, and more preferably from 0.1% to 5% by weight, relative to the total weight of the composition.

The amount of the (c) water in the composition according to the present invention may be from 50% to 95% by weight, preferably from 60% to 90% by weight, and more preferably from 70% to 85% by weight, relative to the total weight of the composition.

The composition according to the present invention may be a cosmetic composition, preferably a skin cosmetic composition, and more preferably a skin care cosmetic composition.

The present invention also relates to a cosmetic process for a keratin substance such as skin, comprising:
applying to the keratin substance the composition according to the present invention; and
drying the composition to form a cosmetic film on the keratin substance.

### BEST MODE FOR CARRYING OUT THE INVENTION

After diligent research, the inventors have discovered that it is possible to provide a composition which is capable of providing an improved moisturizing texture, preferably a combination of an improved moisturizing texture and mattifying effect.

Thus, the composition according to the present invention comprises:
(a) at least one particle, comprising
   at least one cationic polymer and at least one anionic polymer, and
   at least one non-polymeric acid having two or more pKa values or salt(s) thereof;
(b) at least one filler; and
(c) water.

The composition according to the present invention can provide an improved or enhanced moisturizing texture, e.g., a better moist feeling to touch.

The improvement in the moisturizing texture may be attributed to the presence of an (a) particle in the composition according to the present invention.

Also, the composition according to the present invention can reduce or suppress a squeaky texture.

If the filler is oil-absorbing, the composition according to the present invention can capture sebum. Therefore, the composition according to the present invention can reduce gloss on a keratin substance such as skin, and can make, for example, roughness on the skin such as pores and wrinkles less noticeable. Accordingly, the composition according to the present invention can provide optical mattifying effects, while providing an improved moisturizing texture. Thus, the composition according to the present invention can provide both an improved moisturizing texture and optical mattifying effects. The optical mattifying effects would be provided just after the application of the composition according to the present invention and/or would last for long period of time.

Hereinafter, the composition, process, and the like according to the present invention will be explained in a more detailed manner.

[Polyion Complex Particle]

The composition according to the present invention includes (a) at least one particle which is a polyion complex particle. Two or more different types of (a) particles may be used in combination. Thus, a single type of an (a) particle or a combination of different types of (a) particles may be used.

The size of the polyion complex particle may be from 5 nm to 100 μm, preferably from 100 nm to 50 μm, more preferably from 200 nm to 40 μm, and even more preferably from 500 nm to 30 μm. A particle size less than 1 μm can be measured by a dynamic light scattering method, and a particle size more than 1 μm can be measured by an optical microscope. This particle size may be based on number average diameter.

The amount of the (a) particle(s) in the composition according to the present invention may be 0.01% by weight or more, preferably 0.05% by weight or more, and more preferably 0.1% by weight or more, relative to the total weight of the composition.

The amount of the (a) particle(s) in the composition according to the present invention may be 15% by weight or less, preferably 10% by weight or less, and more preferably 5% by weight or less, relative to the total weight of the composition.

The amount of the (a) particle(s) in the composition according to the present invention may be from 0.01% to 15% by weight, preferably from 0.05% to 10% by weight, and more preferably from 0.1% to 5% by weight, relative to the total weight of the composition.

If the composition according to the present invention includes (d) at least one oil explained below, a plurality of the (a) particles can be present at the interface between the (c) water and the (d) oil. Thus, the (a) particles can stabilize an emulsion. For example, if the (c) water constitutes a continuous phase and the (d) oil constitutes dispersed phases, the (a) particles can form an O/W emulsion which may be similar to a so-called Pickering emulsion.

Alternatively, a plurality of the (a) particles can form a capsule having a hollow. The (d) at least one oil can be present in the hollow. In other words, the (d) oil can be incorporated into the capsule. The wall of the capsule may be composed of a continuous layer or film formed from the (a) particles. While not wishing to be bound by theory, it is believed that the (a) particles can re-organize at the interface of the (c) water and the (d) oil to spontaneously form a capsule having a hollow to include the (d) oil. For example, a continuous phase constituted with the (c) water and dispersed phases constituted with the (d) oil in the capsule can form an O/W emulsion which may also be similar to a so-called Pickering emulsion.

The above would mean that the (a) particle itself is amphiphilic. The (a) particle itself is insoluble in oil or water.

The (a) particle includes at least one cationic polymer and at least one anionic polymer.

There is no limit to the type of the cationic and anionic polymers. Two or more different types of cationic polymers may be used in combination. Thus, a single type of cationic polymer or a combination of different types of cationic polymers may be used. Two or more different types of anionic polymers may be used in combination. Thus, a single type of anionic polymer or a combination of different types of anionic polymers may be used.

The ratio of the amount, for example the chemical equivalent, of the cationic polymer(s)/the anionic polymer(s) may be 0.05-18, preferably 0.1-10, and more preferably 0.5-5.0. In particular, it may be preferable that the number of cationic groups of the cationic polymer(s)/the number of anionic groups of the anionic polymer(s) be 0.05-18, more preferably 0.1-10, and even more preferably 0.5-5.0.

The total amount of the cationic and anionic polymers in the composition according to the present invention may be 0.1% by weight or more, preferably 0.5% by weight or more, and more preferably 1% by weight or more, relative to the total weight of the composition.

The total amount of the cationic and anionic polymers in the composition according to the present invention may be 30% by weight or less, preferably 25% by weight or less, and more preferably 20% by weight or less, relative to the total weight of the composition.

The total amount of the cationic and anionic polymers in the composition according to the present invention may be from 0.1% to 30% by weight, preferably from 0.5% to 25% by weight, and more preferably from 1% to 20% by weight, relative to the total weight of the composition.

(Cationic Polymer)

A cationic polymer has a positive charge density. The charge density of the cationic polymer may be from 0.01 meq/g to 20 meq/g, preferably from 0.05 to 15 meq/g, and more preferably from 0.1 to 10 meq/g.

It may be preferable that the molecular weight of the cationic polymer be 500 or more, preferably 1,000 or more, more preferably 2,000 or more, and even more preferably 3,000 or more.

Unless otherwise defined in the descriptions, “molecular weight” means a number average molecular weight.

The cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group. The term (primary) “amino group” here means an —NH₂ group.

The cationic polymer may be a homopolymer or a copolymer. The term “copolymer” is understood to mean both copolymers obtained from two kinds of monomers and those obtained from more than two kinds of monomers, such as terpolymers obtained from three kinds of monomers.

The cationic polymer may be selected from natural and synthetic cationic polymers. Non-limiting examples of the cationic polymers are as follows.

(1) Homopolymers and copolymers derived from acrylic or methacrylic esters and amides and comprising at least one unit chosen from units of the following formulas:

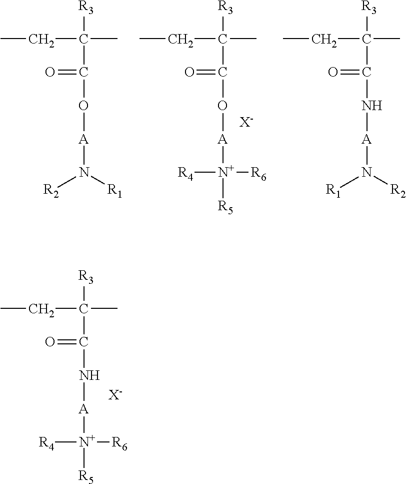

wherein:
R₁ and R₂, which may be identical or different, are chosen from hydrogen and alkyl groups comprising from 1 to 6 carbon atoms, for instance, methyl and ethyl groups;
R₃, which may be identical or different, is chosen from hydrogen and CH₃;
the symbols A, which may be identical or different, are chosen from linear or branched alkyl groups comprising from 1 to 6 carbon atoms, for example, from 2 to 3 carbon atoms and hydroxyalkyl groups comprising from 1 to 4 carbon atoms;
R₄, R₅, and R₆, which may be identical or different, are chosen from alkyl groups comprising from 1 to 18 carbon atoms and benzyl groups, and in at least one embodiment, alkyl groups comprising from 1 to 6 carbon atoms; and
X is an anion derived from an inorganic or organic acid, such as methosulphate anions and halides, for instance chloride and bromide.

The copolymers of family (1) above may also comprise at least one unit derived from comonomers which may be chosen from acrylamides, methacrylamides, diacetone acrylamides, acrylamides and methacrylamides substituted on the nitrogen atom with (C₁-C₄) lower alkyl groups, groups derived from acrylic or methacrylic acids and esters thereof, vinyllactams such as vinylpyrrolidone and vinylcaprolactam, and vinyl esters.

Examples of copolymers of family (1) include, but are not limited to:
copolymers of acrylamide and of dimethylaminoethyl methacrylate quaternized with dimethyl sulphate or with a dimethyl halide,
copolymers of acrylamide and of methacryloyloxyethyltrimethylammonium chloride described, for example, in European Patent Application No. 0 080 976,
copolymers of acrylamide and of methacryloyloxyethyltrimethylammonium methosulphate, quaternized or nonquaternized vinylpyrrolidone/dialkylaminoalkyl acrylate or methacrylate copolymers, described, for example, in French Patent Nos. 2 077 143 and 2 393 573, dimethylaminoethyl methacrylate/vinylcaprolactam/vinylpyrrolidone terpolymers, vinylpyrrolidone/methacrylamidopropyldimethylamine copolymers, quaternized vinylpyrrolidone/dimethylaminopropylmethacrylamide copolymers, and crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with a compound containing an olefinic unsaturation, for example, methylenebisacrylamide.

(2) Cationic cellulose polymers such as cellulose ether derivatives comprising one or more quaternary ammonium groups described, for example, in French Patent No. 1 492 597, such as the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

It is preferable that the cationic cellulose polymer have at least one quaternary ammonium group, preferably a quaternary trialkyl ammonium group, and more preferably a quaternary trimethyl ammonium group.

The quaternary ammonium group may be present in a quaternary ammonium group-containing group which may be represented by the following chemical formula (I):

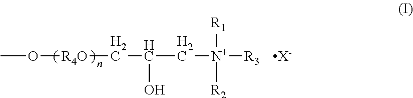

wherein
each of R₁ and R₂ denotes a C₁-C₃ alkyl group, preferably a methyl or ethyl group, and more preferably a methyl group,
R₃ denotes a C₁-C₂₄ alkyl group, preferably a methyl or ethyl group, and more preferably methyl group,
X— denotes an anion, preferably a halide, and more preferably a chloride,
n denotes an integer from 0-30, preferably 0-10, and more preferably 0, and
R₄ denotes a C₁-C₄ alkylene group, preferably an ethylene or propylene group.

The leftmost ether bond (—O—) in the above chemical formula (I) can attach to the sugar ring of the polysaccharide.

It is preferable that the quaternary ammonium group-containing group be —O—CH₂—CH(OH)—CH₂—N⁺(CH₃)₃.

(3) Cationic cellulose polymers such as cellulose copolymers and cellulose derivatives grafted with a water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for example, with a salt chosen from methacryloylethyltrimethylammonium, methacrylamidopropyltrimethylammonium, and dimethyldiallylammonium salts.

Commercial products corresponding to these polymers include, for example, the products sold under the name “Celquat® L 200” and “Celquat® H 100” by the company National Starch.

(4) Non-cellulose-based cationic polysaccharides described in U.S. Pat. Nos. 3,589,578 and 4,031,307, such as guar gums comprising cationic trialkylammonium groups, cationic hyaluronic acid, and dextran hydroxypropyl trimonium chloride. Guar gums modified with a salt, for example the chloride, of 2,3-epoxypropyltrimethylammonium (guar hydroxypropyltrimonium chloride) may also be used.

Such products are sold, for instance, under the trade names JAGUAR® C13 S, JAGUAR® C15, JAGUAR® C17, and JAGUAR® C162 by the company MEYHALL.

(5) Polymers comprising piperazinyl units and divalent alkylene or hydroxyalkylene groups comprising straight or branched chains, optionally interrupted with at least one entity chosen from oxygen, sulphur, nitrogen, aromatic rings, and heterocyclic rings, and also the oxidation and/or quaternization products of these polymers. Such polymers are described, for example, in French Patent Nos. 2 162 025 and 2 280 361.

(6) Water-soluble polyamino amides prepared, for example, by polycondensation of an acidic compound with a polyamine; these polyamino amides possibly being crosslinked with an entity chosen from epihalohydrins; diepoxides; dianhydrides; unsaturated dianhydrides; bisunsaturated derivatives; bishalohydrins; bisazetidiniums; bishaloacyidiamines; bisalkyl halides; oligomers resulting from the reaction of a difunctional compound which is reactive with an entity chosen from bishalohydrins, bisazetidiniums, bishaloacyldiamines, bisalkyl halides, epihalohydrins, diepoxides, and bisunsaturated derivatives; the crosslinking agent being used in an amount ranging from 0.025 to 0.35 mol per amine group of the polyamino amide; these polyamino amides optionally being alkylated or, if they comprise at least one tertiary amine function, they may be quaternized. Such polymers are described, for example, in French Patent Nos. 2 252 840 and 2 368 508.

(7) Polyamino amide derivatives resulting from the condensation of polyalkylene polyamines with polycarboxylic acids, followed by alkylation with difunctional agents, for example, adipic acid/dialkylaminohydroxyalkyldialkylenetriamine polymers in which the alkyl group comprises from 1 to 4 carbon atoms, such as methyl, ethyl, and propyl groups, and the alkylene group comprises from 1 to 4 carbon atoms, such as an ethylene group. Such polymers are described, for instance, in French Patent No. 1 583 363. In at least one embodiment, these derivatives may be chosen from adipic acid/dimethylaminohydroxypropyldiethylenetriamine polymers.

(8) Polymers obtained by reaction of a polyalkylene polyamine comprising two primary amine groups and at least one secondary amine group, with a dicarboxylic acid chosen from diglycolic acid and saturated aliphatic dicarboxylic acids comprising from 3 to 8 carbon atoms. The molar ratio of the polyalkylene polyamine to the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

(9) Cyclopolymers of alkyldiallylamine and cyclopolymers of dialkyldiallyl-ammonium, such as homopolymers and copolymers comprising, as the main constituent of the chain, at least one unit chosen from units of formulas (Ia) and (Ib):

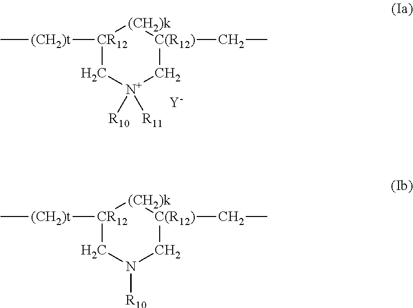

wherein:
k and t, which may be identical or different, are equal to 0 or 1, the sum k+t being equal to 1;
R₁₂ is chosen from hydrogen and methyl groups;
R₁₀ and R₁₁, which may be identical or different, are chosen from alkyl groups comprising from 1 to 6 carbon atoms, hydroxyalkyl groups in which the alkyl group comprises, for example, from 1 to 5 carbon atoms, and lower (C₁-C₄)amidoalkyl groups, or R₁₀ and R₁₁ may form, together with the nitrogen atom to which they are attached, heterocyclic groups such as piperidinyl and morpholinyl; and
Y′ is an anion such as bromide, chloride, acetate, borate, citrate, tartrate, bisulphate, bisulphite, sulphate, and phosphate. These polymers are described, for example, in French Patent No. 2 080 759 and in its Certificate of Addition 2 190 406.

In one embodiment, R₁₀ and R₁₁, which may be identical or different, are chosen from alkyl groups comprising from 1 to 4 carbon atoms.

Examples of such polymers include, but are not limited to, (co)polydiallyldialkyl ammonium chloride such as the dimethyidiallylammonium chloride homopolymer sold under the name “MERQUAT® 100” by the company CALGON (and its homologues of low weight-average molecular mass) and the copolymers of diallyldimethylammonium chloride and of acrylamide sold under the name “MERQUAT® 550”.

Quaternary diammonium polymers comprising at least one repeating unit of formula (II):

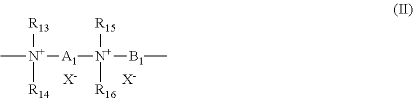

wherein:
R₁₃, R₁₄, R₁₅, and R₁₆, which may be identical or different, are chosen from aliphatic, alicyclic, and arylaliphatic groups comprising from 1 to 20 carbon atoms and lower hydroxyalkyl aliphatic groups, or alternatively R₁₃, R₁₄, R₁₅, and R₁₆ may form, together or separately, with the nitrogen atoms to which they are attached, heterocycles optionally comprising a second heteroatom other than nitrogen, or alternatively R₁₃, R₁₄, R₁₅, and R₁₆, which may be identical or different, are chosen from linear or branched C₁-C₆ alkyl groups substituted with at least one group chosen from nitrile groups, ester groups, acyl groups, amide groups, —CO—O—R₁₇-E groups, and —CO—NH—R₁₇-E groups, wherein R₁₇ is an alkylene group and E is a quaternary ammonium group;
A₁ and B₁, which may be identical or different, are chosen from polymethylene groups comprising from 2 to 20 carbon atoms, which may be linear or branched, saturated or unsaturated, and which may comprise, linked or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from an inorganic or organic acid;
A₁, R₁₃, and R₁₅ may form, together with the two nitrogen atoms to which they are attached, a piperazine ring;
if A₁ is chosen from linear or branched, saturated or unsaturated alkylene or hydroxyalkylene groups, B₁ may be chosen from:

—(CH₂)ₙ—CO-E′-OC—(CH₂)ₙ—
wherein E′ is chosen from:
a) glycol residues of formula —O—Z—O—, wherein Z is chosen from linear or branched hydrocarbon-based groups and groups of the following formulas:

—(CH₂—CH₂—O)ₓ—CH₂—CH₂—

—[CH₂—CH(CH₃)—O]_{y}—CH₂—CH(CH₃)—
wherein x and y, which may be identical or different, are chosen from integers ranging from 1 to 4, which represent a defined and unique degree of polymerization, and numbers ranging from 1 to 4, which represent an average degree of polymerization;
b) bis-secondary diamine residue such as piperazine derivatives;
c) bis-primary diamine residues of formula —NH—Y—NH—, wherein Y is chosen from linear or branched hydrocarbon-based groups and the divalent group —CH₂—CH₂—S—S—CH₂—CH₂—; and
d) ureylene groups of formula —NH—CO—NH—.

In at least one embodiment, X⁻ is an anion such as chloride or bromide.

Polymers of this type are described, for example, in French Patent Nos. 2 320 330; 2 270 846; 2 316 271; 2 336 434; and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

Non-limiting examples of such polymers include those comprising at least one repeating unit of formula (III):

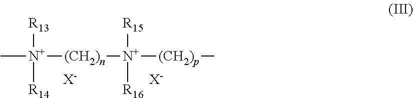

wherein R₁₃, R₁₄, R₁₅, and R₁₆, which may be identical or different, are chosen from alkyl and hydroxyalkyl groups comprising from 1 to 4 carbon atoms, n and p, which may be identical or different, are integers ranging from 2 to 20, and X⁻ is an anion derived from an inorganic or organic acid.

(11) Polyquaternary ammonium polymers comprising units of formula (IV):

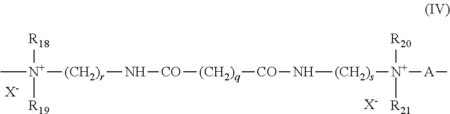

wherein:
R₁₈, R₁₉, R₂₀, and R₂₁, which may be identical or different, are chosen from hydrogen, methyl groups, ethyl groups, propyl groups, p-hydroxyethyl groups, β-hydroxypropyl groups, —CH₂CH₂(OCH₂CH₂)pOH groups, wherein p is chosen from integers ranging from 0 to 6, with the proviso that R₁₈, R₁₉, R₂₀, and R₂₁ are not simultaneously hydrogen,
r and s, which may be identical or different, are chosen from integers ranging from 1 to 6,
q is chosen from integers ranging from 0 to 34,
X⁻ is an anion such as a halide, and
A is chosen from radicals of dihalides and —CH₂—CH₂—O—CH₂—CH₂—.

Such compounds are described, for instance, in European Patent Application No. 0 122 324.

(12) Quaternary polymers of vinylpyrrolidone and of vinylimidazole.

Other examples of suitable cationic polymers include, but are not limited to, cationic proteins and cationic protein hydrolysates, polyalkyleneimines, such as polyethyleneimines, polymers comprising units chosen from vinylpyridine and vinylpyridinium units, condensates of polyamines and of epichlorohydrin, quaternary polyureylenes, and chitin derivatives.

According to one embodiment of the present invention, the at least one cationic polymer is chosen from cellulose ether derivatives comprising quaternary ammonium groups, such as the products sold under the name “JR 400” by the company UNION CARBIDE CORPORATION, cationic cyclopolymers, for instance, the homo-polymers and copolymers of dimethyldiallylammonium chloride sold under the names MERQUAT® 100, MERQUAT® 550, and MERQUAT® S by the company CALGON, guar gums modified with a 2,3-epoxypropyltrimethylammonium salt, and quaternary polymers of vinylpyrrolidone and of vinylimidazole.

(13) Polyamines

As the cationic polymer, it is also possible to use (co)polyamines, which may be homopolymers or copolymers, with a plurality of amino groups. The amino group may be a primary, secondary, tertiary or quaternary amino group. The amino group may be present in a polymer backbone or a pendent group, if present, of the (co)polyamines.

As an example of the (co)polyamines, mention may be made of chitosan, (co)polyallylamines, (co)polyvinylamines, (co)polyanilines, (co)polyvinylimidazoles, (co)polydimethylaminoethylenemethacrylates, (co)polyvinylpyridines such as (co)poly-1-methyl-2-vinylpyridines, (co)polyimines such as (co) polyethyleneimines, (co)polypyridines such as (co)poly(quaternary pyridines), (co)polybiguanides such as (co)polyaminopropyl biguanides, (co)polylysines, (co)polyornithines, (co)polyarginines, (co)polyhistidines, aminodextrans, aminocelluloses, amino(co)polyvinylacetals, and salts thereof.

As the (co)polyamines, it is preferable to use (co)polylysines. Polylysine is well known. Polylysine can be a natural homopolymer of L-lysine that can be produced by bacterial fermentation. For example, polylysine can be s-Poly-L-lysine, typically used as a natural preservative in food products. Polylysine is a polyelectrolyte which is soluble in polar solvents such as water, propylene glycol and glycerol. Polylysine is commercially available in various forms, such as poly D-lysine and poly L-lysine. Polylysine can be in salt and/or solution form.

(14) Cationic Polyaminoacids

As the cationic polymer, it may be possible use cationic polyaminoacids, which may be cationic homopolymers or copolymers, with a plurality of amino groups and carboxyl groups. The amino group may be a primary, secondary, tertiary or quaternary amino group. The amino group may be present in a polymer backbone or a pendent group, if present, of the cationic polyaminoacids. The carboxyl group may be present in a pendent group, if present, of the cationic polyaminoacids.

As examples of the cationic polyaminoacids, mention may be made of cationized collagen, cationized gelatin, steardimonium hydroxypropyl hydrolyzed wheat protein, cocodimonium hydroxypropyl hydrolyzed wheat protein, hydroxypropyltrimonium hydrolyzed conchiolin protein, steardimonium hydroxypropyl hydrolyzed soy protein, hydroxypropyltrimonium hydrolyzed soy protein, cocodimonium hydroxypropyl hydrolyzed soy protein, and the like.

The following descriptions relate to preferable embodiments of the cationic polymer.

It may be preferable that the cationic polymer be selected from cationic starches.

As examples of the cationic starches, mention may be made of starches modified with a 2,3-epoxypropyltrimethylammonium salt (e.g. chloride), such as the product known as starch hydroxypropyltrimonium chloride according to the INCl nomenclature and sold under the name SENSOMER Cl-50 from Ondeo or Pencare™ DP 1015 from Ingredion.

It may also be preferable that the cationic polymer be selected from cationic gums.

The gums may be, for example, selected from the group consisting of cassia gum, karaya gum, konjac gum, gum tragacanth, tara gum, acacia gum and gum arabic.

Examples of cationic gums include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc. or the ClearHance™ from Ashland Inc.

It may be preferable that the cationic polymer be selected from the group consisting of cyclopolymers of alkyldiallylamine and cyclopolymers of dialkyldiallylammonium such as (co)polydiallyldialkyl ammonium chloride, (co)polyamines such as (co)polylysines, cationic (co)polyaminoacids such as cationized collagen, cationic cellulose polymers, and salts thereof.

It may also be preferable that the cationic polymer be selected from chitosans.

It may be more preferable that the cationic polymer be selected from the group consisting of polylysines, polyquaternium-4, polyquaternium-10, polyquaternium-24, polyquaternium-67, starch hydroxypropyl trimonium chloride, cassia hydroxypropyltrimonium chloride, chitosan, and a mixture thereof.

The amount of the cationic polymer(s) in the composition according to the present invention may be 0.01% by weight or more, preferably 0.05% by weight or more, and more preferably 0.1% by weight or more, relative to the total weight of the composition.

The amount of the cationic polymer(s) in the composition according to the present invention may be 15% by weight or less, preferably 10% by weight or less, and more preferably 5% by weight or less, relative to the total weight of the composition.

The amount of the cationic polymer(s) in the composition according to the present invention may be from 0.01% to 15% by weight, preferably from 0.05% to 10% by weight, and more preferably from 0.1% to 5% by weight, relative to the total weight of the composition.

(Anionic Polymer)

An anionic polymer has a positive charge density. The charge density of the anionic polymer may be from 0.1 meq/g to 20 meq/g, preferably from 1 to 15 meq/g, and more preferably from 4 to 10 meq/g if the anionic polymer is a synthetic anionic polymer, and the average substitution degree of the anionic polymer may be from 0.1 to 3.0, preferably from 0.2 to 2.7, and more preferably from 0.3 to 2.5 if the anionic polymer is a natural anionic polymer.

It may be preferable that the molecular weight of the anionic polymer be 300 or more, preferably 1,000 or more, even more preferably 5,000 or more, even more preferably 10,000 or more, even more preferably 50,000 or more, even more preferably 100,000 or more, and even more preferably 1,000,000 or more.

Unless otherwise defined in the descriptions, “molecular weight” may mean a number average molecular weight.

The anionic polymer may have at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a carboxylic group, and a carboxylate group.

The anionic polymer may be a homopolymer or a copolymer. The term “copolymer” is understood to mean both copolymers obtained from two kinds of monomers and those obtained from more than two kinds of monomers, such as terpolymers obtained from three kinds of monomers.

The anionic polymer may be selected from natural and synthetic anionic polymers.

The anionic polymer may comprise at least one hydrophobic chain.

The anionic polymer which may comprise at least one hydrophobic chain may be obtained by copolymerization of a monomer (a) chosen from carboxylic acids comprising an α,β-ethylenic unsaturation (monomer a′) and 2-acrylamido-2-methylpropanesulphonic acid (monomer a″) with a non-surface-active monomer (b) comprising an ethylenic unsaturation other than (a) and/or a monomer (c) comprising an ethylenic unsaturation resulting from the reaction of an acrylic monomer comprising an α,β-monoethylenic unsaturation or of an isocyanate monomer comprising a monoethylenic unsaturation with a monohydric nonionic amphiphilic component or with a primary or secondary fatty amine.

Thus, the anionic polymer with at least one hydrophobic chain may be obtained by two synthetic routes:
either by copolymerization of the monomers (a′) and (c), or (a′), (b) and (c), or (a″) and (c), or (a″), (b) and (c),
or by modification (and in particular esterification or amidation) of a copolymer formed from the monomers (a′) or from the monomers (a′) and (b), or (a″) and (b), by a monohydric nonionic amphiphilic compound or a primary or secondary fatty amine.

Mention may in particular be made, as 2-acrylamido-2-methylpropanesulphonic acid copolymers, of those disclosed in the article “Micelle formation of random copolymers of sodium 2-(acrylamido)-2-methylpropanesulfonate and nonionic surfactant macromonomer in water as studied by fluorescence and dynamic light scattering—Macromolecules, 2000, Vol. 33, No. 10-3694-3704” and in applications EP-A-0 750 899 and EP-A-1 069 172.

The carboxylic acid comprising an α,β-monoethylenic unsaturation constituting the monomer (a′) can be chosen from numerous acids and in particular from acrylic acid, methacrylic acid, crotonic acid, itaconic acid and maleic acid. It is preferably acrylic or methacrylic acid.

The copolymer can comprise a monomer (b) comprising a monoethylenic unsaturation which does not have a surfactant property. The preferred monomers are those which give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The more particularly preferred monomers are methyl acrylate and ethyl acrylate. The other monomers which can be used are, for example, styrene, vinyltoluene, vinyl acetate, acrylonitrile and vinylidene chloride. Unreactive monomers are preferred, these monomers being those in which the single ethylenic group is the only group which is reactive under the polymerization conditions. However, monomers which comprise groups which react under the effect of heat, such as hydroxyethyl acrylate, can optionally be used.

The monomer (c) is obtained by reaction of an acrylic monomer comprising α,β-monoethylenic unsaturation, such as (a), or of an isocyanate monomer comprising monoethylenic unsaturation with a monohydric nonionic amphiphilic compound or a primary or secondary fatty amine.

The monohydric nonionic amphiphilic compounds or the primary or secondary fatty amines used to produce the nonionic monomer (c) are well known. The monohydric nonionic amphiphilic compounds are generally alkoxylated hydrophobic compounds comprising an alkylene oxide forming the hydrophilic part of the molecule. The hydrophobic compounds are generally composed of an aliphatic alcohol or an alkylphenol, in which compounds a carbonaceous chain comprising at least six carbon atoms constitutes the hydrophobic part of the amphiphilic compound.

The preferred monohydric nonionic amphiphilic compounds are compounds having the following formula (V):

R—(OCH₂CHR′)ₘ—(OCH₂CH₂)ₙ—OH (V)

in which R is chosen from alkyl or alkylene groups comprising from 6 to 30 carbon atoms and alkylaryl groups having alkyl radicals comprising from 8 to 30 carbon atoms, R′ is chosen from alkyl groups comprising from 1 to 4 carbon atoms, n is a mean number ranging from approximately 1 to 150 and m is a mean number ranging from approximately 0 to 50, provided that n is at least as great as m.

Preferably, in the compounds of formula (V), the R group is chosen from alkyl groups comprising from 12 to 26 carbon atoms and alkylphenyl groups in which the alkyl group is C₈-C₁₃; the R′ group is the methyl group; m=0 and n=1 to 25.

The preferred primary and secondary fatty amines are composed of one or two alkyl chains comprising from 6 to 30 carbon atoms.

The monomer used to form the nonionic urethane monomer (c) can be chosen from highly varied compounds. Use may be made of any compound comprising a copolymerizable unsaturation, such as an acrylic, methacrylic or allylic unsaturation. The monomer (c) can be obtained in particular from an isocyanate comprising a monoethylenic unsaturation, such as, in particular, α,α-dimethyl-m-isopropenylbenzyl isocyanate.

The monomer (c) can be chosen in particular from acrylates, methacrylates or itaconates of oxyethylenated (1 to 50 EO) C₆-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate or the acrylate modified by polyoxyethylenated (25 EO) C₁₂-C₂₄ alcohols and from dimethyl-m-isopropenylbenzyl isocyanates of oxyethylenated (1 to 50 EO) C₆-C₃₀ fatty alcohol, such as, in particular, the dimethyl-m-isopropenylbenzyl isocyanate of oxyethylenated behenyl alcohol.

According to a specific embodiment of the present invention, the anionic polymer is. chosen from acrylic terpolymers obtained from (a) a carboxylic acid comprising an α,β-ethylenic unsaturation, (b) a non-surface-active monomer comprising an ethylenic unsaturation other than (a), and (c) a nonionic urethane monomer which is the reaction product of a monohydric nonionic amphiphilic compound with an isocyanate comprising a monoethylenic unsaturation.

Mention may in particular be made, as anionic polymers comprising at least one hydrophobic chain, of the acrylic acid/ethyl acrylate/alkyl acrylate terpolymer, such as the product as a 30% aqueous dispersion sold under the name Acusol 823 by Rohm & Haas; the acrylates/steareth-20 methacrylate copolymer, such as the product sold under the name Aculyn 22 by Rohm & Haas; the (meth)acrylic acid/ethyl acrylate/oxyethylenated (25 EO) behenyl methacrylate terpolymer, such as the product as an aqueous emulsion sold under the name Aculyn 28 by Rohm & Haas; the acrylic acid/oxyethylenated (20 EO) monocetyl itaconate copolymer, such as the product as a 30% aqueous dispersion sold under the name Structure 3001 by National Starch; the acrylic acid/oxyethylenated (20 EO) monostearyl itaconate copolymer, such as the product as a 30% aqueous dispersion sold under the name Structure 2001 by National Starch; the acrylates/acrylate modified by polyoxyethylenated (25 EO) C₁₂-C₂₄ alcohol copolymer, such as the 30-32% copolymer latex sold under the name Synthalen W2000 by 3V SA; or the methacrylic acid/methyl acrylate/dimethyl-meta-isopropenylbenzyl isocyanate of ethoxylated behenyl alcohol terpolymer, such as the product as a 24% aqueous dispersion and comprising 40 ethylene oxide groups disclosed in the document EP-A-0 173 109.

The anionic polymers may also be Polyester-5, such as the product sold under the name of Eastman AQ™ 55S Polymer by EASTMAN CHEMICAL having the chemical formula below.

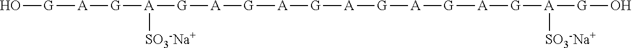

A: dicarboxylic acid moiety
G: glycol moiety
SO₃⁻Na⁺: sodium sulfo group
OH: hydroxyl group

It may be preferable that the anionic polymer be selected from the group consisting of polysaccharides such as alginic acid, hyaluronic acid, and cellulose polymers (e.g., carboxymethylcellulose), anionic (co)polyaminoacids such as (co)polyglutamic acids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers, and salts thereof.

The maleic acid copolymer may comprise one or more maleic acid comonomers, and one or more comonomers chosen from vinyl acetate, vinyl alcohol, vinylpyrrolidone, olefins comprising from 2 to 20 carbon atoms, and styrene.

Thus, the “maleic acid copolymer” is understood to mean any polymer obtained by copolymerization of one or more maleic acid comonomers and of one or more comonomers chosen from vinyl acetate, vinyl alcohol, vinylpyrrolidone, olefins comprising from 2 to 20 carbon atoms, such as octadecene, ethylene, isobutylene, diisobutylene or isooctylene, and styrene, the maleic acid comonomers optionally being partially or completely hydrolysed.

Use will preferably be made of hydrophilic polymers, that is to say polymers having a solubility in water of greater than or equal to 2 g/l.

In an advantageous aspect of the present invention, the maleic acid copolymer may have a molar fraction of maleic acid units of between 0.1 and 1, more preferably between 0.4 and 0.9.

The weight-average molar mass of the maleic acid copolymer may be between 1,000 and 500,000, and preferably between 1,000 and 50,000.

It is preferable that the maleic acid copolymer be a styrene/maleic acid copolymer, and more preferably sodium styrene/maleic acid copolymer.

Use will preferably be made of a copolymer of styrene and of maleic acid in a 50/50 ratio.

Use may be made, for example, of the styrene/maleic acid (50/50) copolymer, in the form of an ammonium salt at 30% in water, sold under the reference SMA1000H® by Cray Valley or the styrene/maleic acid (50/50) copolymer, in the form of a sodium salt at 40% in water, sold under the reference SMA1000HNa® by Cray Valley.

The use of the styrene/maleic acid copolymer such as sodium styrene/maleic acid copolymer may improve the wettability of a film prepared by the composition according to the present invention.

According to one embodiment of the present invention, it is preferable that the anionic polymer be selected from hyaluronic acid and derivatives thereof.

Hyaluronic acid can be represented by the following chemical formula.

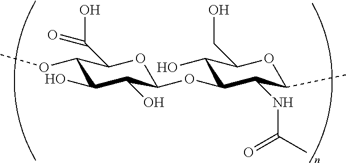

In the context of the present invention, the term “hyaluronic acid” covers in particular the basic unit of hyaluronic acid of formula:

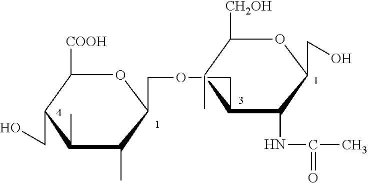

This is the smallest fraction of hyaluronic acid comprising a disaccharide dimer, namely D-glucuronic acid and N-acetylglucosamine.

The term “hyaluronic acid and derivatives thereof” also comprises, in the context of the present invention, the linear polymer comprising the polymeric unit described above, linked together in the chain via alternating P(1,4) and P(1,3) glycosidic linkages, having a molecular weight (MW) that can range between 380 and 13,000,000 daltons. This molecular weight depends in large part on the source from which the hyaluronic acid is obtained and/or on the preparation methods.

The term “hyaluronic acid and derivatives thereof” also comprises, in the context of the present invention, hyaluronic acid salts. As the salts, mention may be made of alkaline metal salts such as sodium salts and potassium salts, alkaline earth metal salts such as magnesium salts, ammonium salts, and mixtures thereof.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid and derivatives thereof” comprises all the fractions or subunits of hyaluronic acid having a molecular weight in particular within the molecular weight range recalled above.

In the context of the present invention, hyaluronic acid fractions which do not have inflammatory activity are preferably used.

By way of illustration of the various hyaluronic acid fractions, reference may be made to the document “Hyaluronan fragments: an information-rich system”, R. Stern et al., European Journal of Cell Biology 58 (2006) 699-715, which reviews the listed biological activities of hyaluronic acid according to its molecular weight.

According to a preferred embodiment of the present invention, the hyaluronic acid fractions suitable for the use covered by the present invention have a molecular weight of between 50,000 and 5,000,000, in particular between 100,000 and 5,000,000, especially between 400,000 and 5,000,000 Da. In this case, the term used is high-molecular-weight hyaluronic acid.

Alternatively, the hyaluronic acid fractions that may also be suitable for the use covered by the present invention have a molecular weight of between 50,000 and 400,000 Da. In this case, the term used is intermediate-molecular-weight hyaluronic acid.

Alternatively again, the hyaluronic acid fractions that may be suitable for the use covered by the present invention have a molecular weight of less than 50,000 Da. In this case, the term used is low-molecular-weight hyaluronic acid.

Finally, the term “hyaluronic acid and derivatives thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The hyaluronic acid derivative may be, for example, acetylated hyaluronic acid or a salt thereof.

The molecular weights indicated above are also valid for the hyaluronic acid esters.

The hyaluronic acid may in particular be hyaluronic acid supplied by the company Hyactive under the trade name CPN (MW: 10 to 150 kDa), by the company Soliance under the trade name Cristalhyal (MW: 1.1.times.10⁶), by the company Bioland under the name Nutra HA (MW: 820 000 Da), by the company Bioland under the name Nutra AF (MW: 69 000 Da), by the company Bioland under the name Oligo HA (MW: 6100 Da) or else by the company Vam Farmacos Metica under the name D Factor (MW: 380 Da).

The amount of the anionic polymer(s) in the composition according to the present invention may be 0.01% by weight or more, preferably 0.05% by weight or more, and more preferably 0.1% by weight or more, relative to the total weight of the composition.

The amount of the anionic polymer(s) in the composition according to the present invention may be 15% by weight or less, preferably 10% by weight or less, and more preferably 5% by weight or less, relative to the total weight of the composition.

The amount of the anionic polymer(s) in the composition according to the present invention may be from 0.01% to 15% by weight, preferably from 0.05% to 10% by weight, and more preferably from 0.1% to 5% by weight, relative to the total weight of the composition.

(Non-Polymeric Acid Having Two or More Acid Dissociation Constants)

The composition according to the present invention may include at least one non-polymeric acid having two or more pKa values or salt(s) thereof, i.e., at least one non-polymeric acid having two or more acid dissociation constants or salt(s) thereof. The pKa value (acid dissociation constant) is well known to those skilled in the art, and should be determined at a constant temperature such as 25° C.

The non-polymeric acid having two or more pKa values or salt(s) thereof can be included in the (a) particle. The non-polymeric acid having two or more pKa values can function as a crosslinker for the anionic polymer and/or the amphoteric polymer.

The term “non-polymeric” here means that the acid is not obtained by polymerizing two or more monomers. Therefore, the non-polymeric acid does not correspond to an acid obtained by polymerizing two or more monomers such as polycarboxylic acid.

It is preferable that the molecular weight of the non-polymeric acid having two or more pKa values or salt(s) thereof is 1,000 or less, preferably 800 or less, and more preferably 700 or less.

There is no limit to the type of the non-polymeric acid having two or more pKa values or salt(s) thereof. Two or more different types of non-polymeric acids having two or more pKa values or salts thereof may be used in combination. Thus, a single type of a non-polymeric acid having two or more pKa values or a salt thereof or a combination of different types of non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metal such as Na and K, and salts with alkaline earth metal such as Mg and Ca, and ammonium salts.

The non-polymeric acid having two or more pKa values or salt(s) thereof may be an organic acid or salt(s) thereof, and preferably a hydrophilic or water-soluble organic acid or salt(s) thereof.

The non-polymeric acid having two or more pKa values may have at least two acid groups selected from the group consisting of a carboxylic group, a sulfuric group, a sulfonic group, a phosphoric group, a phosphonic group, a phenolic hydroxyl group, and a mixture thereof.

The non-polymeric acid having two or more pKa values may be a non-polymeric polyvalent acid.

The non-polymeric acid having two or more pKa values may be selected from the group consisting of dicarboxylic acids, disulfonic acids, and diphosphoric acids, and a mixture thereof.

The non-polymeric acid having two or more pKa values or salt(s) thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, aconitic acid, oxaloacetic acid, tartaric acid, and salts thereof; aspartic acid, glutamic acid, and salts thereof; terephthalylidene dicamphor sulfonic acid or salts thereof (Mexoryl SX), Benzophenone-9; phytic acid, and salts thereof; Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Conc), Green 205 (Light Green SF Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphthol Green B), Green 402 (Guinea Green B), and Black 401 (Naphthol Blue Black); folic acid, ascorbic acid, erythorbic acid, and salts thereof; cystine and salts thereof; EDTA and salts thereof; glycyrrhizin and salts thereof; and a mixture thereof.

It may be preferable that the non-polymeric acid having two or more pKa values or salt(s) thereof be selected from the group consisting of terephthalylidene dicamphor sulfonic acid and salts thereof (Mexoryl SX), Yellow 6 (Sunset Yellow FCF), ascorbic acid, phytic acid and salts thereof, and a mixture thereof.

The amount of the non-polymeric acid having two or more pKa values or salt(s) thereof in the composition according to the present invention may be 0.01% by weight or more, preferably 0.05% by weight or more, and more preferably 0.1% by weight or more, relative to the total weight of the composition.

The amount of the non-polymeric acid having two or more pKa values or salt(s) thereof in the composition according to the present invention may be 15% by weight or less, preferably 10% by weight or less, and more preferably 5% by weight or less, relative to the total weight of the composition.

The amount of the non-polymeric acid having two or more pKa values or salt(s) thereof in the composition according to the present invention may be from 0.01% to 15% by weight, preferably from 0.05% to 10% by weight, and more preferably from 0.1% to 5% by weight, relative to the total weight of the composition.

[Filler]

The composition according to the present invention comprises (b) at least one filler. Two or more fillers may be used in combination. Thus, a single type of filler or a combination of different types of fillers may be used.

The term “filler” should be understood as meaning a colorless or white, inorganic or synthetic particle which is insoluble in a possible liquid component in the composition according to the present invention, whatever the temperature at which the composition is manufactured.

The (b) filler(s) can be inorganic or organic, and can be of spherical or oblong shape, whatever the crystallographic form (for example, sheet, cubic, hexagonal, orthorhombic, and the like). Non-limiting mention may be made of talc, mica, silica, silica silylate, kaolin, sericite, calcinated talc, calcinated mica, calcinated sericite, synthetic mica, bismuth oxychloride, barium sulfate, boron nitride, calcium carbonate, magnesium carbonate, magnesium hydrogen carbonate and hydroxyapatite, powders formed of polyamide (Nylon®), of poly-β-alanine and of polyethylene, powders formed of polyurethane, powders formed of tetrafluoroethylene polymers (Teflon®), lauryllysine, starch, polymeric hollow microspheres, such as those of poly(vinylidene chloride)/acrylonitrile, for example Expancel® (Nobel Industrie), or of acrylic acid copolymers, silicone resin microbeads (Tospearls® from Toshiba, for example), particles formed of polyorganosiloxane elastomers, precipitated calcium carbonate, magnesium carbonate, basic magnesium carbonate, hollow silica microspheres, glass or ceramic microcapsules, or metal soaps derived from organic carboxylic acids having from 8 to 22 carbon atoms, such as from 12 to 18 carbon atoms, for example zinc stearate, magnesium stearate, lithium stearate, zinc laurate or magnesium myristate.

For the present invention, examples of the filler include metal oxides, preferably titanium oxide, zinc oxide and a mixture thereof.

A filler that is suitable for the present invention may be, for example, a filler whose mean particle size is less than 100 μm, and especially between 1 and 50 μm, for example between 4 to 20 μm.

The (b) filler may be selected from hydrophilic or hydrophobic oil-absorbing powders.

The hydrophilic or hydrophobic oil-absorbing powder is capable of absorbing (and/or adsorbing) an oil or a liquid fatty substance, for instance sebum (from the skin).

The hydrophilic or hydrophobic oil-absorbing powder may comprise porous or hollow particles, in particular porous or hollow spherical particles.

(Hydrophilic Oil-Absorbing Powder)

For the purpose of the present invention, the term “hydrophilic” oil-absorbing powder means that said powder (or the particles) is individually dispersed in water in such manner that aggregates are not formed.

The hydrophilic oil-absorbing powder may have an oil-absorbing capacity of 100 ml/100 g or more, preferably 150 ml/100 g or more, and more preferably 200 ml/100 g or more.

The amount of oil absorbed (and/or adsorbed) by the hydrophilic oil-absorbing powder may be characterized by measuring the wet point according to the method described below. The oil-absorbing capacity measured at the wet point, noted Wp, corresponds to the amount of oil that needs to be added to 100 g of powder in order to obtain a homogeneous paste.

The amount of the absorbed (and/or adsorbed) oil can be measured according to the method for determining the oil uptake of a powder described in standard NF T 30-022. It corresponds to the amount of oil absorbed/adsorbed onto the available surface of the powder, by measuring the wet point.

An amount of m=2 g of powder is placed on a glass plate, and an oil (such as ester oil and silicone oil) is then added drop-wise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

Otherwise, the oil-absorbing capacity can be measured in accordance with JIS-K6217-4.

The hydrophilic oil-absorbing powder may be of organic or inorganic nature.

The hydrophilic oil-absorbing powder may be chosen from celluloses, silicas, silicates; perlites; magnesium carbonate; magnesium hydroxide; and derivatives thereof; and a mixture thereof.

According to one embodiment, a cellulose derivative may be chosen from cellulose esters and ethers.

The term “cellulose ester” means, in the text hereinabove and hereinbelow, a polymer consisting of an a (1-4) sequence of partially or totally esterified anhydroglucose rings, the esterification being obtained by reaction of all or only some of the free hydroxyl functions of the said anhydroglucose rings with a linear or branched carboxylic acid or carboxylic acid derivative (acid chloride or acid anhydride) containing from 1 to 4 carbon atoms. Preferably, the cellulose ester results from the reaction of some of the free hydroxyl functions of the said rings with a carboxylic acid containing from 1 to 4 carbon atoms. Advantageously, the cellulose esters are chosen from cellulose acetates, propionates, butyrates, isobutyrates, acetobutyrates and acetopropionates, and mixtures thereof.

The term “cellulose ether” means a polymer consisting of an a (1-4) sequence of partially etherified anhydroglucose rings, some of the free hydroxyl functions of the said rings being substituted with a radical —OR, R preferably being a linear or branched alkyl radical containing from 1 to 4 carbon atoms. The cellulose ethers are thus preferably chosen from cellulose alkyl ethers with an alkyl group containing from 1 to 4 carbon atoms, such as cellulose methyl, propyl, isopropyl, butyl and isobutyl ethers.

Celluloses and their derivatives that may be mentioned include, for example, the following spherical cellulose particles marketed by Daito Kasei in Japan: Cellulobeads USF (oil uptake is 250 ml/100 g) with a particle size of 4 μm (porous cellulose).

Silica powders that may be mentioned include porous silica microspheres, especially those sold under the names Sunsphere® H31 and Sunsphere® H51 (oil uptake equal to 150 ml/100 g) by the company Asahi Glass; MSS-500-3H by the company Kobo; amorphous hollow silica particles, especially those sold under the name Silica Shells by the company Kobo (oil uptake equal to 550 ml/100 g); porous silica microsphere sold under the name of Silysia 350 (oil uptake equal to 310 ml/100 g) by the company Fuji Silysia Chemical; and silica powder sold under the name of Finesil X35 (oil uptake equal to 380 ml/100 g) by the company Oriental Silycas.

A silicate that may especially be mentioned is aluminum silicate which is sold under the name of Kyowaad® 700PEL (oil uptake equal to 195 ml/100 g) by the company Kyowa Chemical Industry.

A perlite powder that may especially be mentioned is the product sold under the name Optimat® 1430 OR and Optimat® 2550 OR by the company World Minerals (oil uptake equal to 240 ml/100 g).

A magnesium carbonate powder that may especially be mentioned is the product sold under the name Tipo Carbomagel® by the company Buschle & Lepper (oil uptake equal to 214 ml/100 g).

A magnesium carbonate/magnesium hydroxide powder that may especially be mentioned is the product of mMgCO₃—Mg(OH)₂-nH₂O which is sold under the name of Mg Tube (oil uptake equal to 250-310 ml/100 g) by the company Nittetsu Mining.

It is preferable that the hydrophilic oil-absorbing powder comprise at least one selected from the group consisting of cellulose, silica, perlite, and a mixture thereof.

(Hydrophobic Oil-Absorbing Powder)

For the purpose of the present invention, the term “hydrophobic” oil-absorbing powder means that said powder (or the particles) is individually dispersed in an oil in such manner that aggregates are not formed.

The hydrophobic oil-absorbing powder may have an oil-absorbing capacity of 100 ml/100 g or more, preferably 150 ml/100 g or more, and more preferably 200 ml/100 g or more.

The amount of oil absorbed (and/or adsorbed) by the hydrophobic oil-absorbing powder may be determined by the method described above.

The hydrophobic oil-absorbing powder may be of organic or inorganic nature.

Organic Hydrophobic Oil-Absorbing Powder:

The organic hydrophobic oil-absorbing powder may be chosen from the group consisting of polyamide (in particular Nylon-6) powders, powders of acrylic polymers, especially of polymethyl methacrylate, of polymethyl methacrylate/ethylene glycol dimethacrylate, of polyallyl methacrylate/ethylene glycol dimethacrylate or of ethylene glycol dimethacrylate/lauryl methacrylate copolymer; and a mixture thereof. The above material may be crosslinked.

It may be preferable that the organic hydrophobic oil-absorbing powder be selected from powders of acrylic polymers, especially of ethylene glycol dimethacrylate/lauryl methacrylate copolymer.

Examples of the organic hydrophobic oil-absorbing powder include the fillers described below.

Acrylic polymer powders that may be mentioned include porous polymethyl methacrylate (INCI name methyl methacrylate crosspolymer) such as the spheres sold under the name Covabead LH85 by the company Sensient, porous polymethyl methacrylate/ethylene glycol dimethacrylate spheres sold under the name Microsponge 5640 by the company Cardinal Health Technologies (oil uptake equal to 155 ml/100 g), ethylene glycol dimethacrylate/lauryl methacrylate crosslinked copolymer powders, especially those sold under the name Polytrap® 6603 from the company Amcol Health & Beauty Solutions (oil uptake equal to 656 ml/100 g), acrylonitrile/methyl methacrylate/vinylidene chloride copolymer sold under the name Expancel 551DE40D42 (oil uptake equal to 1,040 ml/100 g) by the company Akzo Novel.

Polyamide powders that may be mentioned include Nylon-6 powder, especially the product sold under the name Pomp610 by the company UBE Industries (oil uptake equal to 202 ml/100 g).

Inorganic Hydrophobic Oil-Absorbing Powder:

The inorganic hydrophobic oil-absorbing powder may have at least one inorganic core and at least one hydrophobic coating.

It is preferable that the inorganic hydrophobic oil-absorbing powder be selected from powders of hydrophobic silicas, preferably hydrophobic silica aerogels, and more preferably hydrophobic aerogels of silica silylate, and a mixture thereof.

The term “hydrophobic silica” is understood to mean any silica, the surface of which is treated to be hydrophobic.

The hydrophobic silica, in particular silica silylate, may be based on silica aerogels which are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

Aerogels are materials with high porosity. Herein, silica aerogels refer to a solid silica with a porous structure generally obtained by replacing medium included in wet silica gels with air by drying them while a solid network structure of the silica is maintained. The porosity represents the amount of air contained in an apparent volume of a material by a volume percentage. The hydrophobic silica aerogel of the present invention may have a porosity of 60% or more, preferably 70% or more, and more preferably 80% or more.

The hydrophobic silica aerogel particles may exhibit
a specific surface area per unit of weight (SW) ranging from 500 to 1,500 m²/g, preferably from 600 to 1,200 m²/g, and more preferably from 600 to 800 m²/g, and/or a size, expressed as the volume-average diameter (D[0.5]), ranging from 1 to 1,500 μm, preferably from 1 to 1,000 μm, more preferably from 1 to 100 μm, in particular from 1 to 30 μm, more preferably from 5 to 25 μm, more preferably from 5 to 20 μm, and even more preferably from 5 to 15 μm.

The specific surface area per unit of weight can be determined by the nitrogen absorption method, known as the BET (Brunauer-Emmett-Teller) method, described in The Journal of the American Chemical Society, Vol. 60, page 309, February 1938, which corresponds to international standard ISO 5794/1 (appendix D). The BET specific surface area corresponds to the total specific surface area of the particles under consideration.

The sizes of the hydrophobic silica aerogel particles can be measured by static light scattering using a commercial particle size analyzer of MasterSizer 2000 type from Malvern. The data are processed on the basis of the Mie scattering theory. This theory, which is exact for isotropic particles, makes it possible to determine, in the case of non-spherical particles, an “effective” particle diameter. This theory is described in particular in the publication by Van de Hulst, H. C., “Light Scattering by Small Particles”, Chapters 9 and 10, Wiley, New York; 1957.

The hydrophobic silica aerogel particles can advantageously exhibit a packed density (r) ranging from 0.04 g/cm³ to 0.10 g/cm³, and preferably from 0.05 g/cm³ to 0.08 g/cm³.

In the context of the present invention, this density, known as the packed density, can be assessed according to the following protocol:
40 g of powder are poured into a graduated measuring cylinder;
the measuring cylinder is then placed on the Stav 2003 device from Stampf Volumeter;
the measuring cylinder is subsequently subjected to a series of 2500 packing actions (this operation is repeated until the difference in volume between 2 consecutive tests is less than 2%); and
the final volume Vf of packed powder is then measured directly on the measuring cylinder.

The packed density is determined by the ratio w/Vf, in this instance 40/Vf (Vf being expressed in cm³ and w in g).

As regards the preparation of hydrophobic silica aerogel particles modified at the surface by silylation, reference may be made to the document U.S. Pat. No. 7,470,725.

Use will in particular be made of hydrophobic silica aerogel particles modified at the surface with trimethylsilyl groups.

The inorganic hydrophobic oil-absorbing powders that may be mentioned include polydimethylsiloxane-coated amorphous silica microspheres, especially those sold under the name Sunsphere® H33 and Sunsphere® H53 (oil uptake equal to 400 ml/100 g), precipitated silica powders surface-treated with a mineral wax, such as precipitated silica treated with a polyethylene wax, and especially those sold under the name Acematt OR 412 by the company Evonik-Degussa (oil uptake equal to 398 ml/100 g), and silica silylate sold under the name of VM-2270 (oil uptake equal to 1,040 ml/100 g) by the company Dow.

It is preferable to use, as the inorganic hydrophobic oil-absorbing powder, silica silylate sold under the name VM-2270 by Dow, the particles of which exhibit an average size ranging from 5 to 15 μm and a specific surface area per unit of weight ranging from 600 to 800 m²/g.

The hydrophobic silica aerogel particles may be characterized in that the shape of each of the particles is spherical. Due to this spherical shape, the hydrophobic silica aerogel particles can provide cosmetic compositions with good smoothness. The spherical degree of the hydrophobic silica aerogel may be determined by an average circularity.

The spherical hydrophobic silica aerogel particle may have an average circularity of 0.8 or more, and preferably 0.82 or more. The spherical hydrophobic silica aerogel may have an average circularity of less than 1, preferably 0.99 or less, more preferably 0.98 or less, even more preferably 0.97 or less, still even more preferably 0.96 or less, and most preferably 0.95 or less.

The “average circularity” may be determined by an image analysis method. In particular, the “average circularity” may be an arithmetic mean of circularity obtained by image analysis of a scanning electron microscope (SEM) image of no less than 2,000 aerogel particles observed at a magnification of 1,000 by secondary electron detection using a scanning electron microscope (SEM).

The “circularity” of each aerogel particle is a value determined by the following formula:

C=4πS/L²

wherein C represents circularity, S represents the area (projected area) of the aerogel particle in the image, and L represents the length of a periphery (perimeter) of the aerogel particle in the image. When the average circularity approaches 1, the shape of each of the particles becomes more spherical.

The hydrophobic silica aerogel particles that may be used according to the present invention is preferably of silylated silica type (INCI name: silica silylate). Preferably, the hydrophobic silica aerogel particles may be those described in JP-A-2014-088307, JP-A-2014-218433, or JP-A-2018-177620.

It is preferable to use hydrophobic aerogels of silica silylate as the inorganic hydrophobic oil-absorbing powder.

The hydrophobicity of the hydrophobic aerogels of silica silylate may be obtained by reacting a hydrophobicizing agent with a silanol group represented by the following formula existing on the surface of silica:

≡Si—OH

wherein the symbol “a” represents the remaining three valences of the Si atom, thereby converting the silanol group into a group represented by the following formula:

(≡Si—O—)₍₄₋ₙ₎>SiRₙ

wherein n is an integer of 1 to 3; each R is independently a hydrocarbyl group; and two or more R may be the same or different from each other where n is 2 or more.

The hydrophobicizing agent may be a silylating agent. Therefore, according to one preferred embodiment, in the hydrophobic aerogels of silica silylate, the silica particles may be modified at the surface by silylation. As examples of the silylating agents, mention may be made of a treating agent having one of the following formulae (1) to (3).

RₙSiX₍₄₋ₙ₎ Formula (1):

wherein n represents an integer of 1 to 3; R represents a hydrocarbyl group; X represents a group (i.e. a leaving group) which can leave a molecule by cleavage of the bond with the Si atom in a reaction with a compound having a hydroxyl group; each R may be different where n is 2 or more; and each X may be different where n is 2 or less.

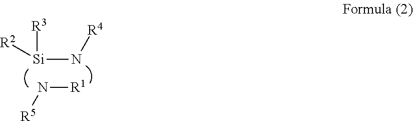

wherein R¹ represents an alkylene group; R² and R³ independently represent a hydrocarbyl group; and R⁴ and R³ independently represent a hydrogen atom or a hydrocarbyl group.

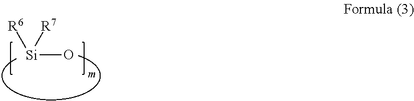

wherein R⁶ and R⁷ independently represent a hydrocarbyl group; m represents an integer of 3 to 6; each R⁶ may be different when there are two or more R⁶; and each R⁷ may be different when there are two or more R⁷.

In the above formula (1), R is a hydrocarbyl group, preferably a hydrocarbyl group having a carbon number of 1 to 10, more preferably a hydrocarbyl group having a carbon number of 1 to 4, and especially preferably a methyl group.

As examples of the leaving group represented by X, mention may be made of halogen atoms such as chlorine and bromine; alkoxy groups such as methoxy group and ethoxy group; groups represented by —NH—SiR₃ (wherein the definition of R is the same as that of R in formula (1)).

Specific examples of the hydrophobicizing agent represented by the above formula (1) include: chlorotrimethylsilane, dichlorodimethylsilane, trichloromethylsilane, monomethyltrimethoxysilane, monomethyltriethoxysilane, and hexamethyldisilazane.

Most preferably, chlorotrimethylsilane, dichlorodimethylsilane, trichloromethylsilane, and/or hexamethyldisilazane may be used from the viewpoint of favorable reactivity.

The number of bonds of the Si atom with the silanol group on the silica framework varies depending on the number (4-n) of the leaving group X. For example, if n is 2, the following bonding will occur:

(≡Si—O—)₂SiR₂.

If n is 3, the following bonding will occur:

≡Si—O—SiR₃.

In this manner, the silanol groups may be silylated, and thereby hydrophobization may be carried out.

In the above formula (2), R¹ may be an alkylene group, preferably an alkylene group having a carbon number of 2 to 8, and especially preferably an alkylene group having a carbon number of 2 to 3.

In the above formula (2), R² and R³ are independently a hydrocarbyl group, and the same preferable groups as those of R in the formula (1) can be mentioned. R⁴ represents a hydrogen atom or a hydrocarbyl group, and when it is a hydrocarbyl group, the same preferable groups as those of R in the formula (1) can be mentioned. When a gel of silica is treated with the compound (cyclic silazane) represented by formula (2), cleavage of Si—N bonds will occur by the reaction with silanol groups, and therefore the following bonding will occur on the surface of the silica framework in the gel:

(≡Si—O—)₂SiR²R³.

In this way, the silanol group may be silylated by the cyclic silazanes of the above formula (2) as well, and thereby hydrophobization may be carried out.

Specific examples of the cyclic silazanes represented by the above formula (3) include hexamethylcyclotrisilazane, and octamethylcyclotetrasilazane.

In the above formula (3), R⁶ and R⁷ are independently a hydrocarbyl group, and the same preferable groups as those of R in the formula (2) can be mentioned. m represents an integer of 3 to 6. When a gel of silica is treated with the compound (cyclic siloxane) represented by the formula (3), the following bonding will occur on the surface of the silica framework in the gel:

(≡Si—O—)₂SiR⁶R⁷.

In this way, silanol groups may be silylated by the cyclic siloxanes of the above formula (3) as well, and thereby hydrophobization may be carried out.

Specific examples of the cyclic siloxanes represented by the above formula (3) include hexamethylcyclotrisiloxane, octamethylcyclotetrasiloxane, and decamethylcyclopentasiloxane.

The hydrophobic aerogels of silica silylate may be prepared by producing a sol of silica, turning the sol into a gel, aging the gel, washing the aged gel, replacing water in the washed gel with a solvent, treating the gel with a hydrophobicizing agent, and dying the hydrophobicized silica.

The hydrophobic aerogels of silica silylate may have a specific surface area determined by BET method of 200 m²/g or more, preferably 400 m²/g or more, and more preferably 500 m²/g or more, and may have a specific surface area determined by BET method of 1,200 m²/g or less, preferably 1,000 m²/g or less, and more preferably 800 m²/g or less.

The hydrophobic aerogels of silica silylate may have a pore volume determined by BJH method of 1 ml/g or more, preferably 2 ml/g or more, and more preferably 3 ml/g or more, and may have a pore volume determined by BJH method of 10 ml/g or less, preferably 8 ml/g or less, and more preferably 7 ml/g or less. The hydrophobic silica aerogel of silica silylate may have a peak pore radius determined by BJH method of 5 nm or more, preferably 10 nm or more, and more preferably 12 nm or more, and may have a peak pore radius determined by BJH method of 50 nm or less, preferably 40 nm or less, and more preferably 30 nm or less.

The “pore volume determined by BJH method” refers to a pore volume which derives from a pore having a pore radius of 1 nm to 100 nm obtained by analyzing, by the BJH method (Barrett, E. P.; Joyner, L. G.; Halenda, P. P., J. Am. Chem. Soc. 73, 373 (1951)), the adsorption isotherm of the nitrogen adsorption side obtained in the same manner as explained in the above “specific surface area determined by BET method”. The “peak pore radius determined by BJT method” refers to a value of a pore radius which gives a peak in a pore distribution curve (volume distribution curve) which is plotted taking on the vertical axis differentiation of the cumulative pore volume by the logarithm of the pore radius obtained by analyzing, by the BJH method, the adsorption isotherm of the nitrogen adsorption side obtained in the same manner as above, and taking the pore radius on the horizontal axis.

The hydrophobic aerogels of silica silylate may have an average particle size of 0.5 μm or more, preferably 1 μm or more, and more preferably 2 μm or more, and may have an average particle size by image analysis method of 30 μm or less, preferably 20 μm or less, and more preferably 15 μm or less.

The “average particle size” here can be measured by image analysis method. Specifically, the value of “average particle size” is an arithmetic mean of equivalent circle diameters which can be obtained by image analysis of a scanning electron microscope (SEM) image of, for example, no less than 2,000 aerogel particles observed at a magnification of 1,000 by secondary electron detection using a scanning electron microscope (SEM). The “equivalent circle diameter” of each aerogel particle is the diameter of a circle having an area equal to the area (projected area) of the aerogel particle in the image.

Preferably, the hydrophobic aerogels of silica silylate may have an oil-absorbing capacity, which can be measured at the wet point, as explained above, of 2 ml/g or more, preferably 3 ml/g or more, more preferably 4 ml/g or more, and most preferably from 5 ml/g or more, and may have an oil-absorbing capacity, measured at the wet point, of 12 ml/g or less, preferably 10 ml/g or less, more preferably 8 ml/g or less, and most preferably 7 ml/g or less.

The amount of the (b) filler(s) in the composition according to the present invention may be 0.01% by weight or more, preferably 0.05% by weight or more, and more preferably 0.1% by weight or more, relative to the total weight of the composition.

The amount of the (b) filler(s) in the composition according to the present invention may be 15% by weight or less, preferably 10% by weight or less, and more preferably 5% by weight or less, relative to the total weight of the composition.

The amount of the (b) filler(s) in the composition according to the present invention may be from 0.01% to 15% by weight, preferably from 0.05% to 10% by weight, and more preferably from 0.1% to 5% by weight, relative to the total weight of the composition.

[Water]

The composition according to the present invention comprises (c) water.

The amount of the (c) water may be 50% by weight or more, preferably 60% by weight or more, and more preferably 70% by weight or more, relative to the total weight of the composition.

The amount of the (c) water may be 95% by weight or less, preferably 90% by weight or less, and more preferably 85% by weight or less, relative to the total weight of the composition.

The amount of the (c) water may be from 50% to 95% by weight, preferably from 60% to 90% by weight, and more preferably from 70% to 85% by weight, relative to the total weight of the composition.

[pH]

The pH of the composition according to the present invention may be from 3 to 9, preferably from 3.3 to 8.5, and more preferably from 3.5 to 8.

At a pH of from 3 to 9, the (a) particle can be very stable.

The pH of the composition according to the present invention may be adjusted by adding at least one alkaline agent and/or at least one acid, other than the non-polymeric acid having two or more pKa values or salt(s) thereof to be incorporated into the (a) particle. The pH of the composition according to the present invention may also be adjusted by adding at least one buffering agent.

(Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

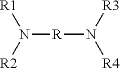

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

(Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as citric acid, lactic acid, sulfuric acid, phosphoric acid and hydrochloric acid (HCl) may be used. HCl is preferable.

The acid(s) may be used in a total amount of from 0.01% to 15% by weight, preferably from 0.02% to 10% by weight, more preferably from 0.03% to 5% by weight, relative to the total weight of the composition, depending on their solubility.

(Buffering Agent)

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris(hydroxymethyl)aminomethane), and a Hepes buffer (4-(2-hydroxyethyl)-1-piperazineethanesulfonic acid).

[Oil]

The composition according to the present invention may comprise (d) at least one oil. If two or more (d) oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, apricot oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) Cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

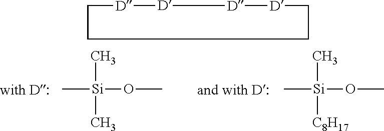

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) Linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics.

The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60,000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

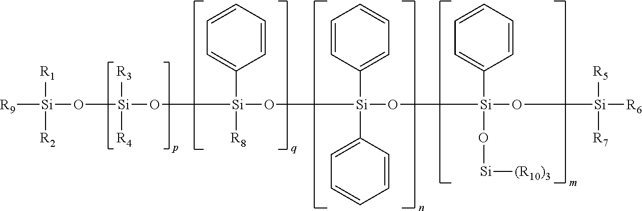

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and
m, n, p and q are, independently of each other, integers of 0 to 900 inclusive, preferably 0 to 500 inclusive, and more preferably 0 to 100 inclusive,
with the proviso that the sum n+m+q is not 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

According to the present invention, the (d) oil may be surrounded by a plurality of the (a) particles or the (d) oil may be present in the hollow of a capsule formed by the (a) particles.

In other words, the (d) oil may be covered by the (a) particles, or a capsule formed by the (a) particles includes the (d) oil in the hollow of the capsule.

The (d) oil which is surrounded by the (a) particles or present in the hollow of the capsule formed by the (a) particles cannot directly make contact with a keratin substance such as skin. Thus, even if the (d) oil has a sticky or greasy feeling of use, the composition according to the present invention would not provide a sticky or greasy feeling of use.

The amount of the (d) oil(s) in the composition according to the present invention may be 0.1% by weight or more, preferably 0.5% by weight or more, and more preferably 1% by weight or more, relative to the total weight of the composition.

The amount of the (d) oil(s) in the composition according to the present invention may be 50% by weight or less, preferably 40% by weight or less, and more preferably 30% by weight or less, relative to the total weight of the composition.

The amount of the (d) oil(s) in the composition according to the present invention may be from 0.1% to 50% by weight, preferably from 0.5% to 40% by weight, and more preferably from 1% to 30% by weight, relative to the total weight of the composition.

[Optional Additives]

The composition according to the present invention may comprise, in addition to the aforementioned components, components typically employed in cosmetics, specifically, surfactants (in particular, nonionic surfactants) or emulsifiers, hydrophilic or lipophilic thickeners, organic volatile or non-volatile solvents, hydrophilic or hydrophobic UV filters, silicones and silicone derivatives other than the (d) oil, natural extracts derived from animals or vegetables, waxes, and the like, within a range which does not impair the effects of the present invention.

The composition according to the present invention may comprise the above optional additive(s) in an amount of from 0.01% to 50% by weight, preferably from 0.05% to 30% by weight, and more preferably from 0.1% to 10% by weight, relative to the total weight of the composition.

[Composition]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, and more preferably a skin care composition.

The composition according to the present invention can be prepared by mixing the above essential and optional ingredients in accordance with any of the processes which are well known to those skilled in the art.

If necessary, the above essential or optional ingredients may be heated. Thus, heating may be performed when mixing the above essential and optional ingredients.

If the composition according to the present invention includes the (d) oil(s), it can be in the form of an emulsion, an O/W emulsion or a W/O emulsion. It is preferable that the composition according to the present invention be in the form of an O/W emulsion because it can provide a fresh sensation due to the (c) water which forms the outer phase thereof.

### [Film]

The composition according to the present invention may be used for easily preparing a film. The (a) particles can aggregate and integrate into a continuous film.

Thus, the present invention may also relate to a process for preparing a film, preferably a cosmetic film, optionally with a thickness of preferably more than 0.1 μm, more preferably 1.5 μm or more, and even more preferably 2 μm or more, comprising:
applying onto a substrate, preferably a keratin substance, more preferably skin, the
composition according to the present invention; and
drying the composition.

The upper limit of the thickness of the above film is not limited. Thus, for example, the thickness of the above film may be 1 mm or less, preferably 500 μm or less, more preferably 300 μm or less, and even more preferably 100 μm or less.

Since the process for preparing a film which may relate to the present invention includes the steps of applying the composition according to the present invention onto a substrate, preferably a keratin substance, and more preferably skin, and of drying the composition, the process does not require any spin coating or spraying, and therefore, it is possible to easily prepare even a relatively thick film. Thus, the process for preparing a film which may relate to the present invention can prepare a relatively thick film without any special equipment such as spin coaters and spraying machines.

Even if the film prepared by the above process is relatively thick, it is still thin and may be transparent, and therefore, may not be easy to perceive. Thus, the film can be used preferably as a cosmetic film.

If the substrate is not a keratin substance such as skin, the composition according to the present invention may be applied onto a substrate made from any material other than keratin.

The materials of the non-keratinous substrate are not limited. Two or more materials may be used in combination. Thus, a single type of material or a combination of different types of materials may be used. In any event, it is preferable that the substrate be flexible or elastic. If the substrate is not a keratin substance, it is preferable that the substrate be water-soluble because it is possible to leave the film by washing the substrate with water. As examples of the water-soluble materials, mention may be made of poly(meth) acrylic acids, polyethyleneglycols, polyacrylamides, polyvinylalcohol (PVA), starch, celluloseacetates, and the like. PVA is preferable.

If the non-keratinous substrate is in the form of a sheet, it may have a thickness of more than that of the film prepared by the above process, in order to ease the handling of the film attached to the substrate sheet. The thickness of the non-keratinous substrate sheet is not limited, but may be from 1 μm to 5 mm, preferably from 10 μm to 1 mm, and more preferably from 50 to 500 μm.

It is more preferable that the film prepared by the above process be releasable from the non-keratinous substrate. The mode of release is not limited. Therefore, the film prepared by the above process may be peeled from the non-keratinous substrate, or released by the dissolution of the substrate sheet into a solvent such as water.

Thus, the present invention may also relate to:
(1) A film, preferably a cosmetic film, optionally with a thickness of preferably more than 0.1 μm, more preferably 1.5 μm or more, and even more preferably 2 μm or more, prepared by a process comprising:
   applying onto a substrate, preferably a keratin substance, and more preferably skin, the composition according to the present invention; and
   drying the composition,
and
(2) A film, preferably a cosmetic film, optionally with a thickness of preferably more than 0.1 μm, more preferably 1.5 μm or more, and even more preferably 2 μm or more, comprising:
   at least one cationic polymer and at least one anionic polymer; and
   at least one non-polymeric acid having two or more pKa values or salt(s) thereof, and
   optionally at least one oil.

The above explanations regarding the cationic and anionic polymers, and the non-polymeric acid having two or more pKa values or salt(s) thereof as well as the above oil can apply to those in the above films (1) and (2).

The film thus obtained above can be self-standing. The term “self-standing” here means that the film can be in the form of a sheet and can be handled as an independent sheet without the assistance of a substrate or support. Thus, the term “self-standing” may have the same meaning as “self-supporting”.

It is preferable that the above film be hydrophobic.

The term “hydrophobic” in the present specification means that the solubility of the polymer in water (preferably with a volume of 1 liter) at from 20° C. to 40° C., preferably from 25° C. to 40° C., and more preferably from 30° C. to 40° C. is less than 10% by weight, preferably less than 5% by weight, more preferably less than 1% by weight, and even more preferably less than 0.1% by weight, relative to the total weight of the polymer. It is most preferable that the polymer is not soluble in water.

If the above film is hydrophobic, the film can have water-resistant properties, and therefore, it can remain on a keratin substance such as skin even if the surface of the keratin substance is wet due to, for example, sweat and rain. Thus, when the film provides any cosmetic effect, the cosmetic effect can last a long time.

On the other hand, the above film can be easily removed from a keratin substance such as skin under alkaline conditions such as a pH of from 8 to 12, preferably from 9 to 11. Therefore, the above film is difficult to remove with water, while it can be easily removed with a soap which can provide such alkaline conditions.

The above film may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself or the metabolism of the microorganisms which may be present in the living body. Also, the biodegradable polymer can be degraded by hydrolysis.

If the above film includes a biocompatible and/or biodegradable polymer, it is less irritable or not irritable to the skin, and does not cause any rash. In addition, due to the use of a biocompatible and/or biodegradable polymer, the above film can adhere well to the skin.

The above film can be used for cosmetic treatments of keratin substances, preferably skin, in particular the face. The above film can be in any shape or form. For example, it can be used as a full-face mask sheet, or a patch for a part of the face such as the cheek, nose, and around the eyes.

If the above film includes at least one hydrophilic or water-soluble UV filter, it can provide UV shielding effects derived from the hydrophilic or water-soluble UV filter. Normally, a hydrophilic or water-soluble UV filter can be removed from the surface of a keratinous substrate such as skin by water such as sweat and rain. However, since the hydrophilic or water-soluble UV filter is included in the above film, it is difficult for the hydrophilic or water-soluble UV filter to be removed by water, thereby resulting in long-lasting UV shielding effects.

[Cosmetic Process and Use]

The present invention also relates to:
a cosmetic process for a keratin substance such as skin, comprising:
applying to the keratin substance the composition according to the present invention; and
drying the composition to form a cosmetic film on the keratin substance, and
a use of the composition according to the present invention for the preparation of a cosmetic film on a keratin substance such as skin.

The cosmetic process here means a non-therapeutic cosmetic method for caring for and/or making up the surface of a keratin substance such as skin.

In both the above process and use, the above cosmetic film is resistant to water with a pH of 7 or less, and is removable with water with a pH of more than 7, preferably 8 or more, and more preferably 9 or more.

In other words, the above cosmetic film can be water-resistant under neutral or acidic conditions such as a pH of 7 or less, preferably in a range of 6 or more and 7 or less, and more preferably in a range of 5 or more and 7 or less, while the above cosmetic film can be removed under alkaline conditions such as a pH of more than 7, preferably 8 or more, and more preferably 9 or more. The upper limit of the pH is preferably 13, more preferably 12, and even more preferably 11.

Accordingly, the above cosmetic film can be water-resistant, and therefore, it can remain on a keratin substance such as skin even if the surface of the keratin substance is wet due to, for example, sweat and rain. On the other hand, the above cosmetic film can be easily removed from a keratin substance such as skin under alkaline conditions. Therefore, the above cosmetic film is difficult to remove with water, while it can be easily removed with a soap which can provide alkaline conditions.

If the above cosmetic film includes a UV filter which may be present in the composition according to the present invention, the above cosmetic film can protect a keratin substance such as skin from UV rays, thereby limiting the darkening of the skin, improving the colour and uniformity of the complexion, and/or treating aging of the skin.

Furthermore, the above cosmetic film may have cosmetic effects such as absorbing or adsorbing malodour and/or protecting the keratin substance from, for example, dirt or pollutants, due to the properties of the polyion complex particles in the cosmetic film, even if the cosmetic film does not include any cosmetic active ingredient.

In addition, the above cosmetic film may immediately change or modify the appearance of the skin by changing light reflection on the skin and the like, even if the cosmetic film does not include any cosmetic active ingredient. Therefore, it may be possible for the above cosmetic film to conceal skin defects such as pores or wrinkles. Further, the above cosmetic film may immediately change or modify the feeling to the touch of the skin by changing the surface roughness on the skin and the like. Furthermore, the above cosmetic film may immediately protect the skin by covering the surface of the skin and shielding the skin, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient other than the (d) oil, the cosmetic film can have cosmetic effects provided by the additional cosmetic active ingredient(s). For example, if the cosmetic film includes at least one cosmetic active ingredient selected from anti-aging agents, anti-sebum agents, deodorant agents, anti-perspirant agents, whitening agents and a mixture thereof, the cosmetic film can treat the aging of the skin, absorbing sebum on the skin, controlling odors on the skin, controlling perspiration on the skin, and/or whitening of the skin.

It is also possible to apply a makeup cosmetic composition onto the above cosmetic film or sheet after it has been applied onto the skin.

### EXAMPLES

The present invention will be described in a more detailed manner by way of examples.

However, they should not be construed as limiting the scope of the present invention.

### Examples 1-11 and Comparative Examples 1-13

[Preparation]

Each of the compositions according to Examples 1-11 and Comparative Examples 1-13 was prepared by mixing the ingredients shown in Tables 1-5 in accordance with the following Steps 1-7.
1. The ingredients for Phase A were mixed and homogenized at 75° C.+/−5° C. to obtain a mixture of Phase A.
2. The ingredient for Phase B was added to the mixture of Phase A and homogenized at 75° C.+/−5° C. to obtain a mixture of Phases A and B.
3. The ingredient for Phase C was added to the above mixture of Phases A and B obtained by Step 2 and homogenized at 75° C.+/−5° C. to obtain a mixture of Phases A, B and C.
4. The ingredients for Phase D were added to the above mixture of Phases A, B and C obtained by Step 3 and homogenized at 75° C.+/−5° C. to obtain a mixture of Phases A, B, C and D.
5. The ingredients for Phase E were mixed and homogenized at 75° C.+/−5° C. to obtain a mixture of Phase E.
6. The mixture of Phase E obtained by Step 5 was added to the above mixture of Phases A, B, C and D obtained by Step 4 and homogenized at 75° C.+/−5° C. to obtain a mixture of Phases A, B, C, D and E.
7. The ingredient for Phase F was added to the above mixture of Phases A, B, C, D and E obtained by Step 6 and homogenized at 75° C.+/−5° C., followed by cooling to room temperature.

The numerical values for the amounts of the ingredients in Tables 1-5 are all based on “% by weight” as active materials.

TABLE 1

| Phase | | Ex. 1 | Ex.2 | Ex. 3 | Ex. 4 | Ex. 5 | Ex. 6 | Ex. 7 | Ex. 8 |
|---|---|---|---|---|---|---|---|---|---|
| A | Water | qsp 100 | qsp 100 | qsp 100 | qsp 100 | qsp 100 | qsp 100 | qsp 100 | qsp 100 |
| | Sodium Hyaluronate | 0.5 | 0.5 | 0.5 | 0.5 | 0.5 | 0.5 | 0.5 | 0.5 |
| B | Polylysine | 0.25 | 0.25 | 0.25 | 0.25 | 0.25 | 0.25 | 0.25 | 0.25 |
| C | Phytic Acid | 0.14 | 0.14 | 0.14 | 0.14 | 0.14 | 0.14 | 0.14 | 0.14 |
| D | Glycerin | 5 | 5 | 5 | 5 | 5 | 5 | 5 | 5 |
| | Butylene Glycol | 3 | 3 | 3 | 3 | 3 | 3 | 3 | 3 |
| | Phenoxyethanol | 0.5 | 0.5 | 0.5 | 0.5 | 0.5 | 0.5 | 0.5 | 0.5 |
| | Chlorphenesin | 0.2 | 0.2 | 0.2 | 0.2 | 0.2 | 0.2 | 0.2 | 0.2 |
| | Dehydroxanthan Gum | 0.5 | 0.5 | 0.5 | 0.5 | 0.5 | 0.5 | 0.5 | 0.5 |
| E | Glyceryl Stearate SE | 1 | 1 | 1 | 1 | 1 | 1 | 1 | 1 |
| | Polyglyceryl-2 Oleate | 0.5 | 0.5 | 0.5 | 0.5 | 0.5 | 0.5 | 0.5 | 0.5 |
| | Polyglyceryl-6 Dicaprate | 1 | 1 | 1 | 1 | 1 | 1 | 1 | 1 |
| | Cetyl Esters (and) Cetyl Esters | 2.5 | 2.5 | 2.5 | 2.5 | 2.5 | 2.5 | 2.5 | 2.5 |
| | Caprylic/Capric Triglyceride | 2.5 | 2.5 | 2.5 | 2.5 | 2.5 | 2.5 | 2.5 | 2.5 |
| | Squalane | 5 | 5 | 5 | 5 | 5 | 5 | 5 | 5 |
| | Behenyl Alcohol | 2 | 2 | 2 | 2 | 2 | 2 | 2 | 2 |
| | Butyrospermum Parki (Shea) Butter | 3 | 3 | 3 | 3 | 3 | 3 | 3 | 3 |
| F | Silica Silylate (Airlica TL-10 from | 0.5 | — | — | — | — | — | — | — |
| | Tokuyama Corp.) | | | | | | | | |
| | Silica Silylate (Dowsil VM-2270 Aerogel | — | 0.5 | — | — | — | — | — | — |
| | Fine Particles from Dow) | | | | | | | | |
| | Silica (Sunsphere H51 from AGC Si-Tech) | — | — | 2 | — | — | — | — | — |
| | Titaniumdioxide/CI 77891 | — | — | — | 2 | — | — | — | — |
| | Cellulose (Cellulobeads USF) | — | — | — | — | 2 | — | — | — |
| | Talc | — | — | — | — | — | 2 | — | — |
| | Perlite | — | — | — | — | — | — | 2 | — |
| | Zinc Oxide | — | — | — | — | — | — | — | 2 |

TABLE 2

| Phase | | Ex. 9 | Ex. 10 | Ex. 11 |
|---|---|---|---|---|
| A | Water | qsp 100 | qsp 100 | qsp 100 |
| | Sodium Hyaluronate | 0.5 | 0.5 | 0.5 |
| B | Polylysine | 0.25 | 0.25 | 0.25 |
| C | Phytic Acid | 0.14 | 0.14 | 0.14 |
| D | Glycerin | 5 | 5 | 5 |
| | Butylene Glycol | 3 | 3 | 3 |
| | Phenoxyethanol | 0.5 | 0.5 | 0.5 |
| | Chlorphenesin | 0.2 | 0.2 | 0.2 |
| | Dehydroxanthan Gum | 0.5 | 0.5 | 0.5 |
| E | Glyceryl Stearate SE | 1 | 1 | 1 |
| | Polyglyceryl-2 Oleate | 0.5 | 0.5 | 0.5 |
| | Polyglyceryl-6 Dicaprate | 1 | 1 | 1 |
| | Cetyl Esters (and) Cetyl Esters | 2.5 | 2.5 | 2.5 |
| | Caprylic/Capric Triglyceride | 2.5 | 2.5 | 2.5 |
| | Squalane | 5 | 5 | 5 |
| | Behenyl Alcohol | 2 | 2 | 2 |
| | Butyrospermum Parki (Shea) Butter | 3 | 3 | 3 |
| F | Silica Silylate (Airlica TL-10 from Tokuyama Corp.) | 2 | — | — |
| | Silica Silylate (Dowsil VM-2270 Aerogel Fine | — | 2 | — |
| | Particles from Dow) | — | | — |
| | Silica (Silica Shells from Kobo) | — | — | 2 |
| | Titaniumdioxide/CI 77891 | — | — | — |
| | Cellulose (Cellulobeads USF) | — | — | — |
| | Talc | — | — | — |
| | Perlite | — | — | — |
| | Zinc Oxide | — | — | — |

TABLE 3

| | | Comp. | Comp. | Comp. | Comp. |
|---|---|---|---|---|---|
| Phase | | Ex. 1 | Ex. 2 | Ex. 3 | Ex. 4 |
| A | Water | qsp 100 | qsp 100 | qsp 100 | qsp 100 |
| | Sodium Hyaluronate | 0.5 | 0.5 | 0.5 | 0.5 |
| B | Polylysine | 0.25 | — | — | — |
| C | Phytic Acid | 0.14 | — | — | — |
| D | Glycerin | 5 | 5 | 5 | 5 |
| | Butylene Glycol | 3 | 3 | 3 | 3 |
| | Phenoxyethanol | 0.5 | 0.5 | 0.5 | 0.5 |
| | Chlorphenesin | 0.2 | 0.2 | 0.2 | 0.2 |
| | Dehydroxanthan Gum | 0.5 | 0.5 | 0.5 | 0.5 |
| E | Glyceryl Stearate SE | 1 | 1 | 1 | 1 |
| | Polyglyceryl-2 Oleate | 0.5 | 0.5 | 0.5 | 0.5 |
| | Polyglyceryl-6 Dicaprate | 1 | 1 | 1 | 1 |
| | Cetyl Esters (and) Cetyl Esters | 2.5 | 2.5 | 2.5 | 2.5 |
| | Caprylic/Capric Triglyceride | 2.5 | 2.5 | 2.5 | 2.5 |
| | Squalane | 5 | 5 | 5 | 5 |
| | Behenyl Alcohol | 2 | 2 | 2 | 2 |
| | Butyrospermum Parki (Shea) Butter | 3 | 3 | 3 | 3 |
| F | Silica Silylate (Airlica TL-10 from Tokuyama Corp.) | — | — | 0.5 | — |
| | Silica Silylate (Dowsil VM-2270 Aerogel Fine | — | — | — | 0.5 |
| | Particles from Dow) | — | — | — | — |
| | Silica (Sunsphere H51 from AGC Si-Tech) | — | — | — | — |
| | Titaniumdioxide/CI 77891 | — | — | — | — |
| | Cellulose (Cellulobeads USF) | — | — | — | — |
| | Talc | — | — | — | — |
| | Perlite | — | — | — | — |
| | Zinc Oxide | — | — | — | — |

TABLE 4

| | | Comp. | Comp. | Comp. | Comp. | Comp. | Comp. |
|---|---|---|---|---|---|---|---|
| Phase | | Ex. 5 | Ex. 6 | Ex. 7 | Ex. 8 | Ex. 9 | Ex. 10 |
| A | Water | qsp 100 | qsp 100 | qsp 100 | qsp 100 | qsp 100 | qsp 100 |
| | Sodium Hyaluronate | 0.5 | 0.5 | 0.5 | 0.5 | 0.5 | 0.5 |
| B | Polylysine | — | — | — | — | — | — |
| C | Phytic Acid | — | — | — | — | — | — |
| D | Glycerin | 5 | 5 | 5 | 5 | 5 | 5 |
| | Butylene Glycol | 3 | 3 | 3 | 3 | 3 | 3 |
| | Phenoxyethanol | 0.5 | 0.5 | 0.5 | 0.5 | 0.5 | 0.5 |
| | Chlorphenesin | 0.2 | 0.2 | 0.2 | 0.2 | 0.2 | 0.2 |
| | Dehydroxanthan Gum | 0.5 | 0.5 | 0.5 | 0.5 | 0.5 | 0.5 |
| E | Glyceryl Stearate SE | 1 | 1 | 1 | 1 | 1 | 1 |
| | Polyglyceryl-2 Oleate | 0.5 | 0.5 | 0.5 | 0.5 | 0.5 | 0.5 |
| | Polyglyceryl-6 Dicaprate | 1 | 1 | 1 | 1 | 1 | 1 |
| | Cetyl Esters (and) Cetyl Esters | 2.5 | 2.5 | 2.5 | 2.5 | 2.5 | 2.5 |
| | Caprylic/Capric Triglyceride | 2.5 | 2.5 | 2.5 | 2.5 | 2.5 | 2.5 |
| | Squalane | 5 | 5 | 5 | 5 | 5 | 5 |
| | Behenyl Alcohol | 2 | 2 | 2 | 2 | 2 | 2 |
| | Butyrospermum Parki (Shea) Butter | 3 | 3 | 3 | 3 | 3 | 3 |
| F | Silica Silylate (Airlica TL-10 from Tokuyama Corp.) | — | — | — | — | — | — |
| | Silica Silylate (Dowsil VM-2270 Aerogel Fine | — | — | — | — | — | — |
| | Particles from Dow) | | | | | | |
| | Silica (Sunsphere H51 from AGC Si-Tech) | 2 | — | — | — | — | — |
| | Titaniumdioxide/CI 77891 | — | 2 | — | — | — | — |
| | Cellulose (Cellulobeads USF) | — | — | 2 | — | — | — |
| | Talc | — | — | — | 2 | — | — |
| | Perlite | — | — | — | — | 2 | — |
| | Zinc Oxide | — | — | — | — | — | 2 |

TABLE 5

| | | Comp. | Comp. | Comp. |
|---|---|---|---|---|
| Phase | | Ex. 11 | Ex. 12 | Ex. 13 |
| A | Water | qsp 100 | qsp 100 | qsp 100 |
| | Sodium Hyaluronate | 0.5 | 0.5 | — |
| B | Polylysine | 0.25 | — | 0.25 |
| C | Phytic Acid | — | 0.14 | 0.14 |
| D | Glycerin | 5 | 5 | 5 |
| | Butylene Glycol | 3 | 3 | 3 |
| | Phenoxyethanol | 0.5 | 0.5 | 0.5 |
| | Chlorphenesin | 0.2 | 0.2 | 0.2 |
| | Dehydroxanthan Gum | 0.5 | 0.5 | 0.5 |
| E | Glyceryl Stearate SE | 1 | 1 | 1 |
| | Polyglyceryl-2 Oleate | 0.5 | 0.5 | 0.5 |
| | Polyglyceryl-6 Dicaprate | 1 | 1 | 1 |
| | Cetyl Esters (and) Cetyl Esters | 2.5 | 2.5 | 2.5 |
| | Caprylic/Capric Triglyceride | 2.5 | 2.5 | 2.5 |
| | Squalane | 5 | 5 | 5 |
| | Behenyl Alcohol | 2 | 2 | 2 |
| | Butyrospermum Parki (Shea) Butter | 3 | 3 | 3 |
| F | Silica Silylate (Airlica TL-10 from Tokuyama Corp.) | — | — | — |
| | Silica Silylate (Dowsil VM-2270 Aerogel Fine | — | — | — |
| | Particles from Dow) | — | — | — |
| | Silica (Sunsphere H51 from AGC Si-Tech) | — | — | — |
| | Titaniumdioxide/CI 77891 | — | — | — |
| | Cellulose (Cellulobeads USF) | — | — | — |
| | Talc | — | — | — |
| | Perlite | — | — | — |
| | Zinc Oxide | — | — | — |

[Evaluations]

(Moisturizing Texture)

Three panelists evaluated the texture, in terms of moisturizing feeling, of each of the compositions according to Examples 1-11 and Comparative Examples 1-13 at the timing of during and after application of the composition. Specifically, each panelist applied each composition on his or her hand and spread it to evaluate moisturizing feeling, and graded from 1 (low) to 5 (high). It was then classified in the following three categories based on the average of the grade:
Good: from 4 to 5
Fair: more than 2 and less than 4
Poor: from 1 to 2

The results are shown in Table 6.

TABLE 6

| | Ex. 1 | Ex. 2 | Ex. 3 | Ex. 4 | Ex. 5 | Ex. 6 | Ex. 7 | Ex. 8 | Ex. 9 | Ex. 10 | Ex. 11 |
|---|---|---|---|---|---|---|---|---|---|---|---|
| Moisturizing Texture | Good | Good | Good | Good | Good | Good | Good | Good | Good | Good | Good |

| | Comp. | Comp. | Comp. | Comp. | Comp. | Comp. | Comp. | Comp. | Comp. | Comp. | Comp. | Comp. | Comp. |
|---|---|---|---|---|---|---|---|---|---|---|---|---|---|
| | Ex. 1 | Ex. 2 | ]Ex. 3 | Ex. 4 | Ex. 5 | Ex. 6 | Ex. 7 | Ex. 8 | Ex. 9 | Ex. 10 | Ex. 11 | Ex. 12 | Ex. 13 |
| Moisturizing Texture | Fair | Poor | Fair | Fair | Poor | Poor | Poor | Fair | Fair | Poor | Poor | Poor | Poor |

Table 6 shows that the composition according to the present invention can provide an improved moisturizing feeling.

On the other hand, Table 6 also shows that the compositions which lack any of the essential ingredients in the composition according to the present invention can provide only limited or relatively inferior moisturizing effects.

(Optical Mattifying Effect)

The mattifying effect of the compositions according to Examples 3, 5 and 9-11 and Comparative Example 1 was evaluated by an in vitro mattifying test. Specifically, each composition was spread over a contrast sheet by an automatic film applicator to form a layer of 100 μm and left to dry at 37° C. for 24 hours. Thereafter, an artificial sebum/sweat composition with the formulation shown in the following Table 7 was sprayed on the above layer on the contrast card, at room temperature. The amount of the artificial sebum/sweat composition sprayed on each of the above layers was the same as each other.

TABLE 7

| | (wt %) |
|---|---|
| Oleic acid | 20.0 |
| Poly(oxy-1,2-ethanediyl) | 1.0 |
| Water | 79.0 |
| Total | 100.0 |

After 6 minutes, the reflectance on the above layer was measured by a glossmeter (GM-268, Konika Minolta) as a 600 gloss value.

The anti-shine index was determined by the following equation:

Anti-Shine Index={(Reflectance on the above layer 6 minutes after the spraying)−(Reflectance of a negative control)}/{(Reflectance of a positive control 6 minutes after the spraying)−(Reflectance of a negative control)}*100

In the above equation, the reflectance was set as 0 for the negative control and 100 for the positive control 6 minutes after the spraying.

The determined anti-shine-index (%) was categorized in accordance with the following criteria:
Excellent: 70 or more
Good: 50 or more and less than 70
Poor: less than 50

The results are shown in Table 8.

TABLE 8

| | | | | | | Comp. |
|---|---|---|---|---|---|---|
| | Ex. 3 | Ex. 5 | Ex. 9 | Ex. 10 | Ex. 11 | Ex. 1 |
| Type of | Silica | Cellulose | Silica | Silica | Silica | — |
| Filler | | | | | | |
| (Amount: | | | Silylate | Silylate | | |
| 2 wt %) | | | | | | |
| Optical | Good | Good | Excellent | Excellent | Good | Poor |
| Mattifying | | | | | | |
| Effect | | | | | | |

Table 8 shows that the composition according to the present invention can provide excellent or good mattifying effect. It is preferable to use silica silylate as the (b) filler to provide excellent mattifying effect.

On the other hand, Table 8 also shows that the composition which lacks oil-absorbing powder as the (b) filler cannot provide a mattifying effect.

1. A composition, comprising:
(a) at least one particle, comprising
at least one cationic polymer and at least one anionic polymer, and
at least one non-polymeric acid having two or more pKa values or salt(s) thereof;
(b) at least one filler; and
(c) water.

2. The composition according to claim 1, wherein the cationic polymer has at least one positively chargeable and/or positively charged moiety selected from the group consisting of a primary, secondary, or tertiary amino group, a quaternary ammonium group, a guanidine group, a biguanide group, an imidazole group, an imino group, and a pyridyl group.

3. The composition according to claim 1, wherein the cationic polymer is selected from the group consisting of cyclopolymers of alkyldiallylamine and cyclopolymers of dialkyldiallylammonium, (co)polyamines, cationic (co)polyaminoacids, cationic cellulose polymers, and salts thereof.

4. The composition according to claim 1, wherein the amount of the cationic polymer(s) forming the (a) particle in the composition is from 0.01% to 15% by weight, relative to the total weight of the composition.

5. The composition according to claim 1, wherein the anionic polymer is selected from hyaluronic acid and derivatives thereof.

6. The composition according to claim 1, wherein the amount of the anionic polymer(s) forming the (a) particle in the composition is from 0.01% to 15% by weight, relative to the total weight of the composition.

7. The composition according to claim 1, wherein the non-polymeric acid having two or more pKa values or salt(s) thereof is an organic acid or salt(s) thereof.

8. The composition according to claim 1, wherein the amount of the non-polymeric acid having two or more pKa values or salt(s) thereof forming the particle in the composition is from 0.01% to 15% by weight, relative to the total weight of the composition.

9. The composition according to claim 1, wherein the amount of the (a) particle in the composition is from 0.01% to 15% by weight, relative to the total weight of the composition.

10. The composition according to claim 1, wherein the (b) filler is selected from hydrophilic or hydrophobic oil-absorbing powders.

11. The composition according to claim 10, wherein the hydrophobic oil-absorbing powders are selected from powders of hydrophobic silicas.

12. The composition according to claim 1, wherein the amount of the (b) filler(s) in the composition is from 0.01% to 15% by weight, relative to the total weight of the composition.

13. The composition according to claim 1, wherein the amount of the (c) water in the composition is from 50% to 95% by weight, relative to the total weight of the composition.

14. The composition according to claim 1, wherein the composition is a cosmetic composition.

15. A cosmetic process for a keratin substance, comprising
applying to the keratin substance the composition according to claim 1; and
drying the composition to form a cosmetic film on the keratin substance.
